SEQUENCE LISTING (58 sequences)

SEQ ID NO: 1 - protein, 710 aa, Artificial Sequence
  NCL
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 2 - protein, 321 aa, Artificial Sequence
  FBRL
MKPGFSPRGGGFGGRGGFGDRGGRGGRGGFGGGRGRGGGFRGRGRGGGGGGGGGGGGGRGGGGFHSGGNRGRGRGGKRGNQSGKNVMVEPHRHEGVFICRGKEDALVTKNLVPGESVYGEKRVSISEGDDKIEYRAWNPFRSKLAAAILGGVDQIHIKPGAKVLYLGAASGTTVSHVSDIVGPDGLVYAVEFSHRSGRDLINLAKKRTNIIPVIEDARHPHKYRMLIAMVDVIFADVAQPDQTRIVALNAHTFLRNGGHFVISIKANCIDSTASAEAVFASEVKKMQQENMKPQEQLTLEPYERDHAVVVGVYRPPPKVKN

SEQ ID NO: 3 - protein, 217 aa, Artificial Sequence
  GAR1
MSFRGGGRGGFNRGGGGGGFNRGGSSNHFRGGGGGGGGGNFRGGGRGGFGRGGGRGGFNKGQDQGPPERVVLLGEFLHPCEDDIVCKCTTDENKVPYFNAPVYLENKEQIGKVDEIFGQLRDFYFSVKLSENMKASSFKKLQKFYIDPYKLLPLQRFLPRPPGEKGPPRGGGRGGRGGGRGGGGRGGGRGGGFRGGRGGGGGGFRGGRGGGFRGRGH

SEQ ID NO: 4 - protein, 48 aa, Artificial Sequence
  NCL-GAR/RGG
GGFGGRGGGRGGFGGRGGGRGGRGGFGGRGRGGFGGRGGFRGGRGGGG

SEQ ID NO: 5 - protein, 40 aa, Artificial Sequence
  FBRL-GAR/RGG
RGGGFGGRGGFGDRGGRGGRGGFGGGRGRGGGFRGRGRGG

SEQ ID NO: 6 - protein, 43 aa, Artificial Sequence
  GAR1-GAR/RGG
RGGGRGGRGGGRGGGGRGGGRGGGFRGGRGGGGGGFRGGRGGG

SEQ ID NO: 7 - protein, 32 aa, Artificial Sequence
  NCL-P1
GGFGGRGGGRGGFGGRGGGRGGRGGFGGRGRG

SEQ ID NO: 8 - protein, 36 aa, Artificial Sequence
  NCL-P2
GGFGGRGGGRGGRGGFGGRGRGGFGGRGGFRGGRGG

SEQ ID NO: 9 - protein, 21 aa, Artificial Sequence
  NCL-P6
RGGFGGRGGGRGGRGGFGGRG

SEQ ID NO: 10 - protein, 22 aa, Artificial Sequence
  FBRL-P1
RGGGFGGRGGFGDRGGRGGRGG

SEQ ID NO: 11 - protein, 21 aa, Artificial Sequence
  FBRL-P2
RGGFGGGRGRGGGFRGRGRGG

SEQ ID NO: 12 - protein, 37 aa, Artificial Sequence
  NCL-P2+G
GGFGGRGGGRGGRGGFGGRGGRGGFGGRGGFRGGRGG

SEQ ID NO: 13 - protein, 39 aa, Artificial Sequence
  NCL-P2+3G
GGFGGRGGGRGGRGGFGGRGGRGGFGGRGGFGGRGGRGG

SEQ ID NO: 14 - protein, 38 aa, Artificial Sequence
  NCL-P2+2G
GGFGGRGGRGGFGGRGGRGGFGGRGGRGGFGGRGGRGG

SEQ ID NO: 15 - protein, 16 aa, Artificial Sequence
  Antennapedia transcription factor penetratin
RQIKIWFQNRRMKWKK

SEQ ID NO: 16 - protein, 11 aa, Artificial Sequence
  HIV protein TAT
YGRKKRRQRRR

SEQ ID NO: 17 - protein, 20 aa, Artificial Sequence
  Head-to-tail repeats in the 48 aminal acid NCL GAR/RGG region
GGFGGRGGGRGGFGGRGGGR

SEQ ID NO: 18 - protein, 19 aa, Artificial Sequence
  Tail-to-tail repeats in the 48 amino acid NCL GAR/RGG region
GGRGGFGGRGRGGFGGRGG

SEQ ID NO: 19 - protein, 9 aa, Artificial Sequence
  Non-repetitive C-terminal region of the 48 amino acid NCL GAR/RGG region
FRGGRGGGG

SEQ ID NO: 20 - protein, 36 aa, Artificial Sequence
  NCL-P2R/K
GGFGGKGGGKGGKGGFGGKGKGGFGGKGGFKGGKGG

SEQ ID NO: 21 - protein, 36 aa, Artificial Sequence
  NCL-P2F/R
GGRGGRGGGRGGRGGRGGRGRGGRGGRGGRRGGRGG

SEQ ID NO: 22 - protein, 36 aa, Artificial Sequence
  NCL-P2R/F
GGFGGFGGGFGGFGGFGGFGFGGFGGFGGFFGGFGG

SEQ ID NO: 23 - protein, 36 aa, Artificial Sequence
  NCL-P2F/Y
GGYGGRGGGRGGRGGYGGRGRGGYGGRGGYRGGRGG

SEQ ID NO: 24 - protein, 36 aa, Artificial Sequence
  NCL-P2F/W
GGWGGRGGGRGGRGGWGGRGRGGWGGRGGWRGGRGG

SEQ ID NO: 25 - protein, 12 aa, Artificial Sequence
  NCL-P3
DHKPQGKKTKFE

SEQ ID NO: 26 - protein, 10 aa, Artificial Sequence
  NCL-P4
RGGFGGRGGG

SEQ ID NO: 27 - protein, 10 aa, Artificial Sequence
  NCL-P7
RGGRGGFGGR

SEQ ID NO: 28 - protein, 13 aa, Artificial Sequence
  CPP1
GRKKRRQRRRPPQ

SEQ ID NO: 29 - protein, 16 aa, Artificial Sequence
  CPP2
RQIKIWFQNRRMKWKK

SEQ ID NO: 30 - protein, 16 aa, Artificial Sequence
  CPP3
RRRRRRRRRRRRRRRR

SEQ ID NO: 31 - protein, 15 aa, Artificial Sequence
  CPP4
RRRRNRTRRNRRRVR

SEQ ID NO: 32 - protein, 18 aa, Artificial Sequence
  CPP5
LLIILRRRIRKQAHAHSK

SEQ ID NO: 33 - protein, 27 aa, Artificial Sequence
  CPP6
GWTLNSAGYLLGKINLKALAALAKKIL

SEQ ID NO: 34 - protein, 22 aa, Artificial Sequence
  CPP7
MVTVLFRRLRIRRACGPPRVRV

SEQ ID NO: 35 - protein, 30 aa, Artificial Sequence
  2xCPP4
RRRRNRTRRNRRRVRRRRRNRTRRNRRRVR

SEQ ID NO: 36 - protein, 13 aa, Artificial Sequence
  CPP4M1
RRNRTRRNRRRVR

SEQ ID NO: 37 - protein, 13 aa, Artificial Sequence
  CPP4M2
RRRRTRRNRRRVR

SEQ ID NO: 38 - protein, 13 aa, Artificial Sequence
  CPP4M3
RRRRNRRNRRRVR

SEQ ID NO: 39 - protein, 13 aa, Artificial Sequence
  CPP4M4
RRRRNRTRRRRVR

SEQ ID NO: 40 - protein, 13 aa, Artificial Sequence
  CPP4M5
RRRRNRTRRNRRR

SEQ ID NO: 41 - protein, 15 aa, Artificial Sequence
  CPP4M6
RRRRNRLRRNRRRLR

SEQ ID NO: 42 - protein, 15 aa, Artificial Sequence
  CPP4M7
RRRRLRLRRLRRRLR

SEQ ID NO: 43 - protein, 15 aa, Artificial Sequence
  CPP4M8
RRRRNRNRRNRRRNR

SEQ ID NO: 44 - protein, 15 aa, Artificial Sequence
  CPP4M9
RRRRRRTRRRRRRVR

SEQ ID NO: 45 - protein, 15 aa, Artificial Sequence
  CPP4M10
RRRRNRRRRNRRRRR

SEQ ID NO: 46 - protein, 60 aa, Artificial Sequence
  GAR/RGG region of NCL (WT)-HA
GGFGGRGGGRGGFGGRGGGRGGRGGFGGRGRGGFGGRGGFRGGRGGGGDHKPQGKKTKFE

SEQ ID NO: 47 - protein, 48 aa, Artificial Sequence; identical to SEQ ID NO: 4
  GAR/RGG region of NCL (698)-HA

SEQ ID NO: 48 - protein, 20 aa, Artificial Sequence
  GAR/RGG region of NCL (670)-HA
GGFGGRGGGRGGFGGRGGGR

SEQ ID NO: 49 - protein, 23 aa, Artificial Sequence
  GAR/RGG region of NCL (652-698)-HA
GGFRGGRGGGGDHKPQGKKTKFE

SEQ ID NO: 50 - protein, 11 aa, Artificial Sequence
  NCL-P5
RGGRGGFGGRG

SEQ ID NO: 51 - protein, 7 aa, Artificial Sequence
  NCL-P7
FRGGRGG

SEQ ID NO: 52 - protein, 19 aa, Artificial Sequence
  FBRL-P3
RGGGGGGGGGGGGRGGGGF

SEQ ID NO: 53 - protein, 37 aa, Artificial Sequence
  NCL-P2+G(F/I)
GGIGGRGGGRGGRGGIGGRGGRGGIGGRGGIRGGRGG

SEQ ID NO: 54 - protein, 37 aa, Artificial Sequence
  NCL-P2+G(F/L)
GGLGRRGGGRGGRGGLGGRGGRGGLGGRGGLRGGRGG

SEQ ID NO: 55 - protein, 37 aa, Artificial Sequence
  NCL-P2+G(G/A)
GGFGARGGARGARGGFGARGARGGFGARGGFRGARGA

SEQ ID NO: 56 - protein, 37 aa, Artificial Sequence
  NCL-P2+G(G/P)
GGFGPRGGPRGPRGGFGPRGPRGGFGPRGGFRGPRGP

SEQ ID NO: 57 - protein, 30 aa, Artificial Sequence
  IPA1E2
GQGQLVKRPKIMRFISHLWLALESANSEKR

SEQ ID NO: 58 - protein, 67 aa, Artificial Sequence
  IPA1E2-P2+G
GQGQLVKRPKIMRFISHLWLALESANSEKRGGFGGRGGGRGGRGGFGGRGGRGGFGGRGGFRGGRGG

(19) United States
(12) Patent Application Publication
(10) Pub. No.: US 2023/0257429 A1
(43) Pub. Date: Aug. 17, 2023

(54) PEPTIDE ADJUVANT FOR ITS THERAPEUTIC APPLICATIONS IN VIRAL AND TUMOUR VACCINE DEVELOPMENT AND CANCER IMMUNOTHERAPY AND AUTOIMMUNE DISEASE DIAGNOSIS AND TREATMENTS

(71) Applicant: National University of Singapore, Singapore (SG)

(72) Inventors: Jinhua Lu, Singapore (SG); Shan Wu, Singapore (SG)

(73) Assignee: National University of Singapore, Singapore (SG)

(21) Appl. No.: 18/016,154

(22) PCT Filed: Jul. 9, 2021
(86) PCT No.: PCT/SG2021/050405

§ 371 Date: Jan. 13, 2023

(30) Foreign Application Priority Data
Jul. 13, 2020 (SG) 10202006656Q

(51) Int. Cl. C07K 14/47; C12N 15/63; A61K 47/64; A61P 29/00

(52) U.S. Cl. CPC C07K 14/47; C12N 15/63; A61K 47/64; A61P 29/00

(57) ABSTRACT

The present invention relates to an isolated peptide, comprising or consisting of a glycine and arginine-rich (GAR/RGG) region with alarmin and/or cell penetrating activity, bioactive fragments or mutants thereof, and compositions comprising the peptide and an antigen or cargo molecule for vaccine development, immunotherapy, and/or the delivery of nucleic acids and proteins into cells. Further, the invention provides a method of detection using these peptides, and a process of producing the peptides.

### FIELD OF THE INVENTION

The present invention relates to an isolated peptide, comprising or consisting of a glycine and arginine-rich (GAR/RGG) region with alarmin and/or cell penetrating activity, bioactive fragments or mutants thereof, and compositions comprising the peptide and an antigen or cargo molecule for vaccine development, immunotherapy, and/or the delivery of nucleic acids, proteins and other cargo into cells. Further, the invention provides a method of detection using these peptides, and a process of producing the peptides.

### BACKGROUND OF THE INVENTION

Multiple tolerance mechanisms guard B cell development and activation against self-antigens [Theofilopoulos, A. N., Kono, D. H. & Baccala, R. Nat Immunol 18: 716-724 (2017); Nemazee, D. Nat Rev Immunol 17: 281-294 (2017)]. However, polyreactive naïve B cells, which react with nuclear antigens, are not uncommon in the naïve repertoire [Wardemann, H. et al., Science 301: 1374-1377 (2003)]. The nucleoli are often targeted by these polyreactive B cell antigen receptors (BCRs) [Wardemann, H. et al., Science 301: 1374-1377 (2003)]. In patients with lupus, rheumatoid arthritis, Sjogren's syndrome, and other systemic and chronic autoimmune diseases, these polyreactive B cells can undergo immunoglobulin class switch and produce pathogenic IgG autoantibodies [Mietzner, B. et al., Proc Natl Acad Sci USA 105: 9729-9732 (2008)]. Another pathway of autoreactive B cell generation is considered to occur through somatic hypermutation in the germinal center [Zhang, J. et al., J Autoimmun 33: 270-274 (2009)]. The nucleoli can be the dominant or the only nuclear regions that are target by patient autoantibodies [Beck, J. S. Lancet 1: 1203-1205 (1961); Nakamura, R. M. & Tan, E. M. Hum Pathol 9: 85-91 (1978)]. Among overall ANA-positive patients, 10-15% produce predominantly anti-nucleolus autoantibodies (ANoA) [Vermeersch, P. & Bossuyt, X. Autoimmun Rev 12: 998-1003 (2013)]. Proteins in the nucleolus are mostly involved in rRNA transcription and processing and ribosome assembly and many are autoantigens [Welting, T. J., Raijmakers, R. & Pruijn, G. J., Autoimmunity Reviews 2: 313-321 (2003); de la Cruz, J., Karbstein, K. & Woolford, J. L. Jr., Annu Rev Biochem 84: 93-129 (2015)]. What confers strong autoimmunogenicity to the nucleolus is not understood, but it inevitably involves the breakdown of B and T cell tolerance and endogenous or exogenous adjuvants. Some autoantigens are components of ribonucleoproteins (RNP) in which the RNA components exhibit adjuvant activities through activation of Toll-like Receptors (TLR) [Suurmond, J. & Diamond, B., J Clin Invest 125: 2194-2202 (2015)]. An ANoA-specific B cell clone has been reported to seed primary autoimmune germinal centers in which other autoreactive B cells expand to produce broader autoantibody specificities [Degn, S. E. et al., Cell 170: 913-926 2017].

The mammalian immune system encompasses an innate arm that captures and senses common pathogen-associated molecular patterns (PAMPs) and an adaptive arm that profiles the antigenic epitopes in the same microbes. How the innate arm is activated by a pathogen fundamentally affects how the adaptive arm processes and responds to the epitopes giving rise to tailored B and T cell immunity and immunological memory [Pulendran, B. & Ahmed, R., Cell 124: 849-863 (2006)]. Extracellular bacterial and fungal infections induce antibodies that activate complement and Fc receptors to kill and eradicate these pathogens. Intracellular viral infections are associated with both extracellular and intracellular antigen presentation leading to both antibody production and CD8 cytotoxic T lymphocyte (CTL) activation that respectively block viral infection and eradicate the viruses through killing infected cells [Blum, J S., Wearsch, P. A. & Cresswell, P., Annu Rev Immunol 31: 443-473 (2013)]. Cancer cells accumulate neoepitopes that are specific targets of immune surveillance and these are most productively targeted by CTLs [Hollingsworth, R E. & Jansen, K. NPJ Vaccines 4: 7 (2019); Chen, F. et al. J Clin Invest 129: 2056-2070 (2019)].

Dozens of pathogens have been attenuated, inactivated or fractionated as pathogen mimicries or vaccines and optimized empirically to induce immune responses and immunological memories without causing the diseases that the pathogens usually cause (https://www.cdc.gov/vaccines/vpd/vaccines-list.html). However, production and safety concerns have excluded many pathogens from conventional vaccine production. In this context, viral surface proteins often contain adequate MHC class I and II epitopes that can elicit protective T and B cell activation against the pathogens e.g. SARS-CoV2 [Grifoni, A. et al., Cell Host Microbe 27: 670-680 (2020); Ahmed, S. F., Quadeer, A. A. & McKay, M. R., Viruses 12 (2020)]. Natural viral infection yields cytoplasmic antigens that are presented through MHC I to activate CD8 T cells into CTLs [Blum, J S., Wearsch, P. A. & Cresswell, P., Annu Rev Immunol 31: 443-473 (2013)]. Live viruses also harbor adjuvants that activate APCs through one or more innate immune receptors such as Toll-like receptors (TLRs) [Duthie, M S., Windish, H. P., Fox, C. B. & Reed, S. G., Immunol Rev 239: 178-196 (2011); Steinhagen, F., Kinjo, T., Bode, C. & Kinman, D. M., Vaccine 29: 3341-3355 (2011)]. Outside the viral context, single viral protein vaccine antigens lack cytoplasmic access and are not known to have intrinsic adjuvant signals. Cancer antigens are intracellular antigens that are most effectively presented through MHC I and best targeted by CTLs [Blum, J S., Wearsch, P. A. & Cresswell, P., Annuv Rev Immunol 31: 443-473 (2013)]. In the empirical preparation of vaccines, these are often compensated by including surrogate adjuvants in the vaccine compositions. The scarcity of effective recombinant protein vaccines in use for viral pathogens and cancers stresses the need of innovative adjuvants that enable these protein antigens to display their antigenicity [Coffman, R L., Sher, A. & Seder, R. A., Immunity 33: 492-503 (2010); Lee, S. & Nguyen, M T., Immune Netw 15: 51-57 (2015)].

Here we report a group of peptides with alarmin and/or cell-penetrating activities that may be used as adjuvants in vaccines and/or as carriers of cargo molecules into cells.

### SUMMARY OF THE INVENTION

The present invention provides peptides with alarmin and/or cell-penetrating activities for vaccine development, immunotherapy, drug delivery, and diagnosis of inflammation. Alarmins cause the activation of antigen-presenting cells such as monocytes, macrophages and dendritic cells. Nucleolin (NCL) is the most prominent protein autoantigen in severe SLE patients who exhibit elevated TLR7 polymorphism, especially in male patients [Wang, T. et al., Front Immunol 10: 1243 2019], and it is also known to induce autoantibodies early in lupus-prone mice before they develop other autoantibodies and lupus-like diseases [Hirata, D. et al., Clin Immunol 97: 50-58 (2000)]. Our hypothesis was that some autoantigens are autoimmunogenic because they carry alarmin activity. Therefore, we examined whether NCL also contains alarmin activity and discovered an alarmin peptide within it. We then further discovered that the peptide and its mutation variants also exhibit cell-penetrating activities. Therefore, we discovered a group of related peptides that contain alarmin and/or cell penetrating activities.

According to a first aspect, the present invention provides an isolated peptide comprising or consisting of a glycine and arginine-rich (GAR/RGG) region with alarmin and/or cell penetrating activity.

In some embodiments, the glycine and arginine-rich (GAR/RGG) region of the peptide comprises or consists of a plurality of amino acid trimers selected from the group comprising RGG, GGR, FGG and GGF.

In some embodiments, the glycine and arginine-rich (GAR/RGG) region of the peptide further comprises tetramers selected from the group comprising RGGG, GGGR, FGGG and GGGF and/or intervening amino acids selected from the group comprising RG, GR, FR and GDR.

In some embodiments, the peptide is selected from the group comprising or consisting of NCL (SEQ ID NO: 1), FBRL (SEQ ID NO: 2), GAR1 (SEQ ID NO: 3), or an alarmin-active and/or cell penetrating fragment or mutant thereof.

In some embodiments, the peptide comprises or consists of an amino acid sequence set forth in the group comprising or consisting of;

NCL-GAR/RGG:
(SEQ ID NO: 4)
GGFGGRGGGRGGFGGRGGGRGGRGGFGGRGRGGFGGRGGFRGGRGGGG;
FBRL-GAR/RGG:
(SEQ ID NO: 5)
RGGGFGGRGGFGDRGGRGGRGGFGGGRGRGGGFRGRGRGG;
GAR1-GAR/RGG:
(SEQ ID NO: 6)
RGGGRGGRGGGRGGGGRGGGRGGGFRGGRGGGGGGFRGGRGGG,
and
NCL(698)-HA, where the GAR\RGG comprises:
(SEQ ID NO: 47)
GGFGGRGGGRGGFGGRGGGRGGRGGFGGRGRGGFGGRGGFRGGRGGGG,

or an alarmin-active and/or cell penetrating fragment or mutant thereof.

In some embodiments a peptide mutant comprises one or more amino acid additions or deletions, such as the addition of one or more ‘G’ residues. Advantageously, the mutant peptide comprises an insertion of one or more ‘G’ residues within the GAR/RGG region to complete a triplet, such as “RGRGG” to “RGGRGG”, or “RGGFRGG” to “RGGFGGRGG”.

In some embodiments, the peptide comprises or consists of an amino acid sequence set forth in the group comprising or consisting of;

NCL-P1:
(SEQ ID NO: 7)
GGFGGRGGGRGGFGGRGGGRGGRGGFGGRGRG;
NCL-P2:
(SEQ ID NO: 8)
GGFGGRGGGRGGRGGFGGRGRGGFGGRGGFRGGRGG;
NCL-P6:
(SEQ ID NO: 9)
RGGFGGRGGGRGGRGGFGGRG;
FBRL-P1:
(SEQ ID NO: 10)
RGGGFGGRGGFGDRGGRGGRGG,
and
FBRL-P2:
(SEQ ID NO: 11)
RGGFGGGRGRGGGFRGRGRGG

In some embodiments, the mutant peptide comprises or consists of an amino acid sequence set forth in the group comprising or consisting of;

NCL-P2 + G:
(SEQ ID NO: 12)
GGFGGRGGGRGGRGGFGGRGGRGGFGGRGGFRGGRGG;
NCL-P2 + 3G:
(SEQ ID NO: 13)
GGFGGRGGGRGGRGGFGGRGGRGGFGGRGGFGGRGGRGG,
NCL-P2 + 2G:
(SEQ ID NO: 14)
GGFGGRGGRGGFGGRGGRGGFGGRGGRGGFGGRGGRGG.
NCL-P2R/K:
(SEQ ID NO: 20)
GGFGGKGGGKGGKGGFGGKGKGGFGGKGGFKGGKGG,
NCL-P2F/R:
(SEQ ID NO: 21)
GGRGGRGGGRGGRGGRGGRGRGGRGGRGGRRGGRGG,
NCL-P2R/F:
(SEQ ID NO: 22)
GGFGGFGGGFGGFGGFGGFGFGGFGGFGGFFGGFGG,
NCL-P2F/Y:
(SEQ ID NO: 23)
GGYGGRGGGRGGRGGYGGRGRGGYGGRGGYRGGRGG
NCL-P2F/W:
(SEQ ID NO: 24)
GGWGGRGGGRGGRGGWGGRGRGGWGGRGGWRGGRGG;
NCL-P2 + G(F/I):
(SEQ ID NO: 53)
GGIGGRGGGRGGRGGIGGRGGRGGIGGRGGIRGGRGG;
NCL-P2 + G(F/L):
(SEQ ID NO: 54)
GGLGGRGGGRGGRGGLGGRGGRGGLGGRGGLRGGRGG;
NCL-P2 + G(G/A):
(SEQ ID NO: 55)
GGFGARGGARGARGGFGARGARGGFGARGGFRGARGA;
and
NCL-P2 + G(G/P):
(SEQ ID NO: 56)
GGFGPRGGPRGPRGGFGPRGPRGGFGPRGGFRGPRGP.

In some embodiments, the peptide or mutant thereof has both alarmin activity and cell-penetrating activity.

In some embodiments, the peptide with alarmin activity and cell-penetrating activity consists of an amino acid sequence set forth in the group comprising SEQ ID NO: 7, SEQ ID NO: 8, SEQ ID NO: 12, SEQ ID NO: 13, SEQ ID NO: 53 and SEQ ID NO: 54.

In some embodiments, the peptide has cell-penetrating activity and diminished alarmin activity. The peptide mutants NCL-P2F/R (SEQ ID NO: 21), NCL-P2F/Y (SEQ ID NO: 23) and NCL-P2F/W (SEQ ID NO: 24) have cell-penetrating activity but no significant alarmin activity and are useful as carriers of cargo molecules.

The peptide may have adjuvant and/or carrier function. Peptides with alarmin activity also act as adjuvants, these terms being used interchangeably in the context of the present invention.

In some embodiments, the peptide of the invention is fused to an antigen or cargo molecule.

Fusion of an antigen to a peptide having adjuvant activity is advantageous for vaccine development. Fusion of the peptide of the invention to a peptide, such as a peptide antigen may be described as a fusion polypeptide.

It would be understood that fusion includes known means for conjugating or joining the peptides and peptide mutants of the invention to an antigen or cargo molecule, respectively. Such fusion could be generated through recombinant DNA methods, peptide synthesis, or chemical conjugation.

In some embodiments, the peptide can penetrate cells and carry an antigen or cargo molecule into said cells. In some embodiments the peptide and antigen are not fused together but in admixture in a composition. Preferably, the cells are dendritic cells or other antigen-presenting cells, or T cells.

In some embodiments, the at least one antigen is specific to a pathogen, such as a bacterium, fungus, parasite or virus, or to a cancer cell. In some embodiments, the at least one antigen is a virus protein.

In some embodiments, the cargo molecule is a drug or labelling molecule.

According to a second aspect, the present invention provides a composition comprising:

a) an isolated peptide of any aspect of the invention, and at least one antigen; or

b) an isolated fusion polypeptide of any aspect of the invention; or

c) an inactivated cancer cell and a peptide of any aspect of the invention,

and one or more of a pharmaceutically acceptable excipient, diluent or carrier, or a mixture thereof.

In a previous study, a surrogate antigen (ovalbumin) was transfected to express in the T lymphoblast EL4 cells (ATCC TIB-39). When these cells were injected into syngeneic mice, it induced ovalbumin-specific cytotoxic T lymphocytes in the mice that killed the EL4-OVA cells [Moore, M. W., et al., Cell, 54(6): Pages 777-785 (1988)]. In this study, it is unclear whether the injected EL4-OVA cells functioned as antigen-presenting cells or cancer cells. Without being bound by theory, it is proposed that if cancer cells are transfected to express the peptides of the invention with or without additional cancer antigens and then, after inactivation, injected as vaccines, the peptides may make these cancer cells effective cancer vaccines. Alternatively, the peptides could be simply penetrated into cancer cells to make them immunogenic (i.e. induce immunity against the antigens already inside the cancer cells).

In some embodiments, the composition is a vaccine composition.

According to a third aspect, the present invention provides a method of enhancing the immunogenicity of an antigen, wherein the antigen is specific to a pathogen, such as a bacterium, fungus, parasite or virus, or to a cancer cell, comprising fusing or mixing a peptide alarmin of the invention with said antigen.

According to a fourth aspect, the present invention provides a use of an isolated peptide, fusion polypeptide or composition of any aspect of the invention for the manufacture of a medicament for the prophylaxis or treatment of a disease, wherein the disease is a viral, fungal, parasitic, bacterial or cancer disease.

In some embodiments, the medicament comprises an isolated peptide comprising a peptide alarmin having an amino acid sequence selected from the group comprising SEQ ID NO: 12, SEQ ID NO: 13, SEQ ID NO: 53 and SEQ ID NO: 54.

In some embodiments, the medicament comprises an isolated peptide comprising a peptide alarmin having an amino acid sequence selected from the group comprising SEQ ID NO: 12, SEQ ID NO: 13, SEQ ID NO: 53 and SEQ ID NO: 54 fused to an antigen or cargo molecule.

According to a fifth aspect, the present invention provides a method of prophylaxis or treatment of a subject in need of such treatment, comprising administering to the subject:

a) an isolated alarmin peptide of the invention fused to or mixed with an antigen or cargo molecule; or

b) a composition comprising same.

In some embodiments, the present invention provides a method of prophylaxis or treatment of a subject, comprising administering to the subject the peptide alarmin of the invention fused to an immune checkpoint or other polypeptide biological that targets tumour cells. Preferably, the peptide adjuvant is selected from the group comprising SEQ ID NO: 12, SEQ ID NO: 13, SEQ ID NO: 53 and SEQ ID NO: 54.

One application of the alarmin and cell-penetrating activity of the peptide of the invention is to activate T cells. This can be achieved through activation/penetration of dendritic cells but these peptides can also directly prime or activate T cells because they also express alarmin receptors for these peptides. For example, T cell activation is shown in FIG. 24. Since T cells express the peptide receptor (FIGS. 13 D and H), it's possible that the peptides also directly stimulate T cells to synergize with dendritic cells in T cell activation. Further, the peptide may be used to activate T cells or even B (FIGS. 24 C and G) cells directly based on FIG. 24. T cells can also have antigen-presenting capacity.

According to a sixth aspect, the present invention provides a method of activating at least one dendritic cell or other antigen presenting cell, or a T cell, comprising exposing said at least one dendritic cell, antigen presenting cell or T cell to an isolated peptide of any aspect of the invention, or the isolated peptide fused or mixed with an antigen or cargo molecule.

According to a seventh aspect, the present invention provides an isolated polynucleotide encoding the peptide or fusion polypeptide of any aspect of the invention.

As will be appreciated by those of skill in the art, in certain embodiments, the nucleic acid may further comprise a plasmid sequence. The plasmid sequence can include, for example, one or more sequences of a promoter sequence, a selection marker sequence, or a locus-targeting sequence. Methods of introducing nucleic acid compositions into cells are well known in the art.

According to an eighth aspect of the invention there is provided a cloning or expression vector comprising one or more polynucleotides encoding a peptide or fusion polypeptide of the invention operably linked to a promoter.

According to a ninth aspect, the present invention provides a process for the production of a peptide or fusion polypeptide of any aspect of the invention, comprising culturing a host cell, or cell-free polypeptide manufacturing composition, comprising an expression vector comprising one or more polynucleotides encoding said peptide or fusion polypeptide of the invention operably linked to a promoter and isolating the respective peptide or fusion polypeptide.

In some embodiments the fusion polypeptide comprises an NCL-P2+G alarmin/adjuvant peptide and an antigen such as potential cancer antigen peptide IPA1E2. In some embodiments IPA1E2 comprises the amino acid sequence set forth in SEQ ID NO: 57. In some embodiments the amino acid sequence of the NCL-P2+G-IPA1E2 fusion polypeptide is set forth in SEQ ID NO: 58.

According to a tenth aspect, the present invention provides a method for detecting GAR/RGG-containing peptides in a subject, comprising the steps;

i) providing a biological sample from said subject;
ii) determining a level of GAR/RGG-containing proteins present in said biological sample.

In some embodiments, the subject has an autoimmune disease, wherein a level of GAR/RGG-containing peptides above a control level indicates an autoimmune disease in the subject.

In some embodiments, the subject has been administered an isolated peptide or fusion polypeptide or composition of the invention.

In some embodiments, the method comprises contacting the sample in i) with an antibody specific for a GAR/RGG-containing protein. Preferably, the antibody binds specifically to a GAR/RGG region of said GAR/RGG-containing peptide, such as nucleolin (NCL), fibrillarin (FBRL), or GAR1, or bioactive GAR/RGG region mutants thereof.

In some embodiments, the biological sample is selected from the group comprising blood, cerebrospinal fluid and urine.

According to an eleventh aspect, the present invention provides a method of enhancing the intracellular delivery of an antigen or cargo molecule, such as a nucleic acid or polypeptide reagent or therapeutic drug, for the purpose of research or disease treatment, comprising the combination of a peptide of the invention with said antigen or cargo molecule.

The inventors have identified a potent adjuvant (alarmin) and/or cell penetrating activity carried by a short peptide and its mutants. Peptide alarmins are rare and peptides with both alarmin and cell penetrating activities are unique. The GAR/RGG peptide may be included in a composition containing a vaccine antigen, especially a viral or cancer vaccine antigen, to enhance their immunogenicity.

The GAR/RGG peptide is not only found in the nucleolar protein nucleolin (NCL), but also in many other nuclear autoantigens. It is a linear and aqueously soluble peptide without significant secondary structures or cytotoxicity, which makes it a perfect linking peptide for multiple vaccine antigens.

Application of the GAR/RGG peptide in vaccine development is a positive application of an otherwise detrimental pathophysiological phenomenon. This application intends to transfer intrinsic adjuvant activities of autoantigens to vaccines but not their antigenicity. The NCL GAR/RGG sequence does not significantly contribute epitopes based on antigenicity prediction and ELISA using P2+G-coated plates to screen SLE patient autoantibodies (data not shown).

The GAR/RGG peptide of NCL has dual adjuvant properties: 1) it can activate TLR2 which is expressed on APCs and some lymphocytes and 2) it can also penetrate the cell membrane so it is expected to deliver vaccine antigens into the cytoplasm of APCs in fusion or separately added forms. Recombinant protein antigens are much simpler and safer to produce than attenuated/inactivated whole pathogens, but they have rarely been made into successful vaccines, notwithstanding the recent use of mRNA vaccines to produce recombinant coronavirus spike proteins. The key reasons are 1) their low immunogenicity/efficacy and 2) their inaccessibility to the APC cytoplasm to induce CTL immunity, which is indispensable for effective immune defense against virus, cancer and other intracellular pathogens. The ability of the GAR/RGG peptide of the invention to penetrate the cell membrane as well as activate APCs can effectively compensate these weaknesses found in recombinant protein antigens and potentially enable a new generation of cheap and safe vaccines for many diseases.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### BRIEF DESCRIPTION OF THE FIGURES

FIGS. 1A-J show that nucleolin is a potent alarmin that activates PBMC, monocytes, macrophages and dendritic cells (DC). A) isolation of nucleolin and other proteins to stimulate blood leucocytes. HeLa cells were homogenized to isolate nuclei by centrifugation through 2.2 M sucrose. Nuclei were depleted of lipid envelopes with Triton X-100 which we name as T×N. With T×N, nuclear materials were extracted from the chromatin fibers using 0.5 M NaCl and these extracted nuclear materials are known as T×NE. NCL and HMGB1 were isolated from T×NE by affinity chromatography. T×NE was also applied onto a non-immune mouse IgG1 column and equivalent elution fractions 1-3 were pooled as a control (Ms IgG1). Fraction 10 eluted from these columns lack detectable proteins and these were combined as another control (E10). All stimulants and controls were coated on the plate to stimulate the different cells. LPS was used as a positive control. Cell activation was determined by measuring TNFα and IL-1β secretion into the culture media. B and C) TNFα and IL-1β induced from PBMC. D and E) TNFα and IL-1β induced from monocytes. F and G) TNFα induced from DC and macrophages, respectively. Triple experiments were performed and data were presented as mean±SD. **** p<0.0001. n.d. not detectable. H-J) Kinetics of TNFα and IL-1β induction from PBMCs. PBMC were stimulated for 2.5, 5.0, 10, 14, 18 and 24 hr with NCL (H), HMGB1 (I) or LPS (J). TNFα and IL-1β were measured in the culture media. Note the similar kinetics of cytokine induction by NCL and HMGB1.

FIGS. 2A-B show the level of endotoxin in purified nuclear proteins. NCL and HMGB1 were affinity-purified using mouse anti-NCL and mouse anti-HMGB1 antibodies that were cross-linked to Protein G-Sepharose. NCL-HA and its deletion mutants were affinity-purified using mouse anti-HA antibody cross-linked to Protein G-Sepharose. The proteins were first dialyzed into PBS and then diluted to 40 μg/ml. Before being coated on the plates, endotoxin was measured in these proteins using the ToxinSensor chromogenic LAL endotoxin assay kit (GenScript). Similar assays were performed with other purified proteins used in this study. The levels of endotoxins detected were typically below 0.1 EU/μg (A) or 0.5 EU/ml (B).

FIGS. 3A-E show that nucleolin activates TLR2. A) Schematic illustration of an NF-κB luciferase assay used to examine NCL activation of TLRs. The extracellular ligand-binding domains of TLRs contain leucine-rich repeats. TLR4 functions in complex with MD2 and CD14. TLR2 functions in homodimers or in heterodimers with TLR1, TLR6 or TLR10. TLR5 functions independent of co-receptors. A known ligand for each of these TLRs has been indicated. Their cytoplasmic domain interacts with MyD88 to cause PI3 kinase (PI3K), MAPK and NF-κB activation leading to cell activation and cytokine production. In this assay, NF-κB activation is measured by co-transfection of 293T cells with TLRs and two luciferase-expressing plasmids: inducible firefly luciferase expression controlled under five repeats of NF-κB promoter sequences (5XNF-κB) as a measure of TLR signaling and constitutive Renilla luciferase expression under the control of the CMV promoter for normalizing cell numbers and transfection efficiencies of different experiments. B) Roles of TLRs and inflammasome in NCL activation of PBMC. PBMCs were pre-incubated for 1 hr with the MyD88 inhibitor st-2825 (30 μm), the caspase 1 inhibitor (10 μm), or both before culturing for 24 hr with NCL, HMGB1 or LPS (0.5 μg/ml). As a control, cells were preincubated with DMSO before stimulation. TNFα and IL-1β were measured in the media. C and D) NF-κB luciferase assay. 293T cells were transfected with the NF-κB firefly and CMV Renilla luciferase expression vectors and co-transfected with TLR2, TLR4, TLR5, MD2 and CD14 expression vectors as indicated. After 24 hr, cells were harvested and re-cultured in NCL- or HMGB1-coated plates. As a control, these cells were cultured with LPS (0.5 μg/ml). Luciferase activities were measured using the Dual-Luciferase® Reporter Assay System. NF-κB activation was derived by normalizing the firefly luciferase activity to Renilla luciferase activity in each experiment. Triplicate experiments were performed and data were presented as mean±SD. Statistics was performed by one-way ANOVA. **** p<0.0001; *** p<0.001, ** p<0.01, * p<0.05. E) Role of TLR2, TLR4 and TLR5 in PBMC responses to NCL stimulation. PBMC were pre-incubated for 30 min on ice with mouse monoclonal antibodies, known to block TLR2, TLR4 or TLR5 response to their respective ligands, and then cultured for 24 hr either in plates coated with NCL or HMGB1 or, as controls, cultured in blank plates with LTA (10 μg/ml), flagellin (1 μg/ml) or LPS (10 ng/ml) stimulation. The activation of PBMC was measured based on TNFα production. Experiments were performed in triplicates and student t test was performed. * p<0.05, ** p<0.01, *** p<0.001, **** p<0.0001.

FIGS. 4A-F show IL-1β-dependent and -independent TNFα induction by NCL (A, D) as well as HMGB1 (B, E) and LPS (C, F). Monocytes were stimulated with NCL, HMGB1 and LPS in the presence of either a neutralizing anti-IL-1β antibody or non-immune mouse IgG. After 24 hr, TNFα (A-C) and IL-1β (D-F) were determined in the cultures by ELISA. Experiments were performed in triplicates with means and standard deviations being presented. Statistics was performed by student t test. * p<0.05, ** p<0.01. *** p<0.001, ****p<0.0001.

FIGS. 5A-B show titration of MyD88 inhibitor st-2825 and caspase 1 inhibitor Ac-YVAD. Monocytes were pre-incubated with the inhibitors for 1 hr at a serial of indicated concentrations and then stimulated for 24 hr with LPS. TNFα (A) and IL-1β (B) production was measured by ELISA and cell viability was determined using the colourimetric MTS assay. Data was expressed as relative cell viability taking the controls as 1.0. Experiments were performed in triplicates and student t test was performed. * p<0.05, **p<0.01.

FIG. 6 shows selective TLR2 activation by NCL. 293T cells were transfected with the NF-κB promoter-regulated firefly and CMV promoter-directed Renilla luciferase expression vectors. Cells were selectively co-transfected with the TLR4/MD2/CD14, TLR2/1/6/10, TLR5, or TLR3/7/8/9 vectors as indicated. After 24 hr, the transfected cells were harvested and re-cultured in plates coated with NCL or, as a control, the elution from the mouse IgG1 column (Ms IgG). Cells were stimulated for 24 hr and NF-κB-mediated luciferase activities were determined using the Dual-Luciferase Reporter Assay System (Promega). Relative NF-κB activation was derived by normalizing the firefly luciferase activity in each experiment against the constitutive Renilla luciferase activity. Triplicate experiments were performed and data were presented as mean±SD. Statistics was performed by one-way ANOVA. **** p<0.0001; ** p<0.01.

FIGS. 7A-F show the identification of TLR2-reactive regions on NCL. A) Recombinant NCL generated with different domain deletions. Full-length HMGB1 and NCL were expressed each with a C-terminal HA tag (HMGB1-HA and NCL-HA). NCL is a 710-amino acid long and serial C-terminal deletions were made, with reference to boundaries of the acidic, RRM1, RRM2, RRM3 RRM4, and glycine- and arginine-rich (GAR) or RGG domains, to generate NCL mutants that contain, counting from the N-terminus, 274, 477, 522, 609, 649, 670 and 698 residues. The GAR/RGG domain contained two tandem repeats (black boxes) and two reverse repeats (grey boxes). A mutant was also generated by deleting these four repeats spanning residues 653-698. B) Purified NCL-HA and NCL mutants were coated on the plates (40 μg/ml) to culture with isolated monocytes. After 24 hr, TNFα and IL-β1 production was determined by ELISA. C) Purified NCL, NCL-HA, NCL(649)-HA and BSA (10 μg/ml) were coated on the plates and then incubated with increasing concentrations of His-tagged TLR2 (0.375-6.0 μg/ml). Bound TLR2 was detected using a mouse anti-His antibody (2.6 μg/ml). D) TLR2 (2 μg/ml) was coated on the plates and then incubated with purified NCL, NCL-HA, NCL(649)-HA or BSA at increasing concentrations (0-20 μg/ml). Bound proteins were detected using a mouse anti-HA antibody (1 μg/ml). E) TLR2 (2 μg/ml) was coated on the plates and then incubated with anti-TLR2 or anti-TLR4 antibodies (5 μg/ml) before incubation with purified NCL, NCL-HA and BSA (10 μg/ml). Bound proteins were detected using rabbit anti-NCL antibodies (1 μg/ml). F) In a summary experiment, NCL, seven NCL-HA mutants and, as a control, BSA were coated on the plates (10 μg/ml). After incubation with TLR2 (2 μg/ml), bound TLR2 was detected using a mouse anti-His antibody (2.6 μg/ml). * p<0.05, *** p<0.001, **** p<0.0001.

FIGS. 8A-G show synthetic peptides corresponding to the GAR/RGG domain in NCL (SEQ ID NO: 46) are recognized by TLR2 and activate monocytes through TLR2. A) Six peptides were synthesized based on the GAR/RGG sequence of NCL with N-terminal biotin. As a control, the NCL C-terminal peptide of 12 amino acid residues were also synthesized. B) TLR2 was coated on the plates (2 μg/ml) and incubated with NCL-P1, NCL-P2 and NCL-P3. NCL-P1 and NCL-P2 cover 46 residues in the entire 48-residue GAR/RGG region of NCL and overlap in the middle 20 residues. NCL-P3 corresponds to the NCL C-terminus and it lacked TLR2 binding. C) NCL-P1 and NCL-P2 activate TLR2-mediated NF-κB activation. 293T cells were transfected for 24 hr with combinations of TLR4/CD14/MD2, TLR2/TLR1/TLR6/TLR10, TLRS or TLR3/7/8/9 and co-transfected with a vector encoding for firefly luciferase under the inducible NF-κB promoter and a vector for Renilla luciferase under the constitutively active CMV promoter. Cells were then stimulated for 24 hr with the peptides (200 μg/ml). Normalized firefly luciferase activity was used to indicate NF-κB activation. D) Monocytes were cultured for 24 hr with each of the three NCL peptides at different concentrations and TNFα production was measured in the supernatants by ELISA. E) Peptides were coated on the plates at 10, 40 and 160 μg/ml. The plates were used to stimulate monocytes for 24 hr. TNFα production was determined by ELISA. n.d., not detectable. The coated peptides are less stimulatory than the soluble peptides. F) TLR2 was coated on the plates (2 μg/ml) and incubated with different concentrations of a total of 5 NCL peptides. NCL-P2 was included as a positive control. NCL-P4, NCL-P5 and NCL-P6 were peptides covering shorter regions within NCL-P2. NCL-P7 corresponds to the last 7 residues of NCL-P2. BSA was used as a negative control. G) Monocytes were stimulated for 24 hr with 7 different NCL peptides. Peptides were added to the monocyte culture at 50 or 200 μg/ml and TNFα production was determined by ELISA.

FIG. 9 shows coated NCL-HA is more potent than soluble NCL-HA in monocyte stimulation. Monocytes (1×10⁵/well) were cultured for 24 hr in plates. NCL-HA (40 μg/ml) was either pre-coated on the plate or added in its soluble form to culture with monocytes. In addition, monocytes were also cultured in plate coated with 5-fold less NCL (0.2×NCL-HA). Buffer control, wells coated with buffer. Cell alone, wells that were not coated. TNFα and IL-1β were determined in the culture supernatants by ELISA. Experiments were performed in triplicates and presented as mean±SD. Statistics was performed by one-way ANOVA. * p<0.05, ** p<0.01. *** p<0.001, **** p<0.0001, n.s., not significant.

FIG. 10 shows monocyte activation by NCL peptides. A total of seven NCL peptides, as detailed in FIG. 8A, were used to stimulate monocytes at two different concentrations (50 and 200 μg/ml). After 24 hr, IL-1β was determined in the culture media by ELISA.

FIGS. 11A-E show the alarmin activity of fibrillarin (FBRL) and GAR1. A) Amino acid sequence of FBRL with the GAR/RGG sequence motifs being highlighted in bold. The three peptides that were synthesized are also indicated using overlines (FBRL-P1 and FBRL-P2) and underline (FBRL-P3) and the overlapping residues are in grey. FBRL contains two GAR regions but only the long GAR region close to the N-terminus was investigated. It was deleted to generate the FBRL(Δ8-64)-HA mutant. B) Data obtained with PBMC from two different blood donors are shown. FBRL-HA and FBRL(Δ8-64)-HA were separately coated on the plates (10 μg/ml) to stimulate PBMC for 24 hr before ELISA measurement of TNFα in the media. C) PBMC were cultured for 24 hr after addition of FBRL peptides to 50 or 160 mg/ml. Measurement of TNFα in the media was made by ELISA. D) Amino acid sequence of GAR1 with the GAR motifs being highlighted in bold. A recombinant GAR1 was generated with a C-terminal HA tag (GAR1-HA). E) Recombinant GAR1-HA was purified and coated on the plate to stimulate PBMC. TNFα production was determined by ELISA. Control, cells cultured without coated proteins or added peptides. Triplicate experiments were performed to obtain data as mean±SD. Data was analyzed by one-way ANOVA. * p<0.05, p<0.0001, n.s., not significant.

FIGS. 12A-B shows the release of NCL and compares NCL isolated from the nuclear extract (T×NE) and NCL released by UV-induced cells in the activation of monocytes. A) HeLa cells were cultured in 150-mm dishes and UV-irradiated in serum-free media as previously described (Cai et al., 2017, incorporated herein by reference). Cells were, after UV-irradiation, cultured under the same condition for 0-24 hr and media were harvested either immediately (0 hr) or after 1, 3, 6, 8, 12 or 24 hr. After passing through 0.22-μm filters, the media were analyzed by SDS-PAGE (12.5% (w/v)) and Western blotting to monitor the release of nuclear proteins. NPM1, nucleophosmin-1. FBL, fibrillarin (FBRL). B) NCL was affinity-purified from the 24-hr culture medium of UV-irradiated cells (NCL UV sup) and compared with NCL affinity-purified from T×NE (NCL T×NE). Proteins were coated on the plates to stimulate monocytes. As controls, wells were coated with the protein-free elution fraction 10 (E10). Cells were also cultured in uncoated (cell alone) wells with or without LPS stimulation. After 24 hr, TNFα and IL-1β production were determined by ELISA. Experiments were performed in triplicates and presented as mean±SD. Statistics was performed by one-way ANOVA. ** p<0.01, **** p<0.0001, ns: not significant.

FIG. 13 shows incubation of the 36-AA GAR/RGG peptide (P2) with PBMC, monocytes, B cells and T cells all led to high intracellular peptide pools at 4° C. or 37° C. PBMCs were incubated with the biotin-P2 peptide for 1 hr at 37° C. Initially, the incubation was also performed at 4° C. as a control. Cells were then incubated with anti-CD14 (monocytes, BV711), anti-CD3 (T cells, PerCP-Cy5-5), anti-CD19 (B cells, Pacific blue), and the Zombie NIR Cell Viability reagent (APC-Cy7, Biolegend). Cells were then incubated with streptavidin Alex Fluor 488 (streptavidin-AF488) to detect surface-bound P2 peptide. Cell were also fixed/permeabilized with the Fix/Perm reagent (ThermoFisher, Waltham, Mass.) before incubation with streptavidin-AF488 to detect intracellular P2 peptide, which was initially only expected at 37° C. incubation. Cells were, after washing, analyzed by flow cytometry. Note that, while surface binding was only prominent on PBMC and monocytes, all cells exhibited similarly high levels of intracellular P2 peptide irrespective of the temperature of incubation. Dotted histograms, signals detected without membrane permeabilization (surface peptide). Solid histograms, signals detected with membrane permeabilization (intracellular peptide).

FIG. 14 shows the P1 and P2 but not the other shorter GAR/RGG peptides (P4-P7; 8-20 AA), not the R to K mutant of P2 (P2R/K), or not a 12-AA non-GAR/RGG peptide (P3) accumulate intracellular pools after incubation with PBMCs at 4° C. Briefly, PBMCs were incubated separately with each biotin-labeled peptide (P1-P7) or the biotin-P2(R/K) mutant for 1 hr 4° C. Cells were then fixed/permeabilized with the Fix/Perm reagent and incubated with streptavidin-AF488. After washing, cells were analyzed by flow cytometry. Left panel, histograms obtained after cells were incubated with each of the 7 peptides. Right panel, only histograms generated with the NCL-P1 and NCL-P2 peptides are shown.

FIG. 15 shows a schematic explanation of immunity induced through natural viral infection and that induced by P2-fused vaccine antigens. Such fusions can be generated through recombinant DNA method or chemical linkers such as EMCS (N-ε-malemidocaproyl-oxysuccinimide ester). Here ‘P2’ or ‘*’ represents all bioactive GAR/RGG peptides included in claims 1-13. The ‘virus’ image represents all pathogens and cancer cells from which vaccine antigens can be derived. The half circle image attached with ‘P2’ or ‘*’ can be cargo drugs, labels as well as vaccine antigens. The attachment can be direct fusion or indirect mixing. The large ‘cell’ image can be antigen-presenting cells or any other cell types depending the cargo that is fused with the ‘P2’ peptide. TCR, T cell antigen receptor. BCR, B cell antigen receptor. TLR, Toll-like receptor. FcR, Fc receptor. MHC, major histocompatibility complex. CTL, cytotoxic T lymphocytes. Black dotted lines, cytokines.

FIGS. 16A-C shows eight sequence variants of the NCL-P2 peptide and their gain or loss of adjuvant activity. A) Sequences of the NCL-P2 sequence variants. NCL-P6 and the FBRL peptides are included as reference experiments. In NCL-P2R/K (SEQ ID NO: 20), all R residues were changed to K residues. In NCL-P2F/R (SEQ ID NO: 21), all F residues were changed to R residues. In NCL-P2R/F (SEQ ID NO: 22), all R residues were changed to F residues. In NCL-P2F/Y (SEQ ID NO: 23), all F residues were changed to Y residues. In NCL-P2F/W (SEQ ID NO: 24), all F residues were changed to W residues. In NCL-P2+G (SEQ ID NO: 12), an additional G residue was added to change a ‘RG’ sequence to a RGG′ sequence. In NCL-P2+3G, two more G residues were added to change a ‘FRGG’ sequence to a ‘FGGRGG’ sequence. In NCL-P2+2G, the NCL-P2 sequence were streamlined to have virtually four repeats of FGGRGGRGG sequences. Although NCL-P2R/F (SEQ ID NO: 22) was designed, it has not been synthesized. B and C) The NCL-P2, its variant peptides, and the three FBRL peptides were compared to stimulate PBMCs for 24 hr and TNFα was measured in the media. B and C) The NCL-P2 variant peptides were used to stimulate PBMCs for 24 hr and TNFα was measured in the media.

FIGS. 17A-H show cell-penetrating peptide (CPP) activities of NCL-P2 and its seven variant peptides. PBMC (100 μl; 3×10⁵/ml) were incubated with each peptide (200 μg/ml) for 1 hr at 4° C. Cells were washed twice in 2% FBS/PBS and incubated with streptavidin-AF488 (50 μg/ml) and Zombie (NIR) Fixable viability stain-APC-Cy7 for 30 min at 4° C. After washing, cells were analysed by flow cytometry to detect surface-bound peptides (Sur-). To detect intracellular peptides, cells were, after incubation with the peptides at 4° C., incubated with the Zombie NIR Cell Viability reagent. Cells were washed and permeablized with BD CYTOFIX/CYTOPERM™ Kit for 20 min at 4° C. After washing, cells were incubated with streptavidin-AF488 and analysed by flow cytometry to detect intracellular peptide (Int-). Without prior incubation with peptides, cells were incubated with streptavidin-AF488 as controls (shaded histograms). (A) NCL-P2, (B) NCL-P2R/K, (C) NCL-P2F/R, (D) NCL-P2F/Y, (E) NCL-P2F/W, (F) NCL-P2+G, (G) NCL-P2+2G, and (H) NCL-P2+3G. Cells incubated with peptide and streptavidin-AF488 are shown as open histograms. Vertical lines, positions of histogram for surface-bound and intracellular NCL-P2.

FIGS. 18A-B shows micrographs of the kinetics of dendritic cell (DC) penetration by P2+G (P2M6) and its streptavidin conjugates. (A) DC on coverslips were incubated with P2+G (200 μg/ml) on ice for up to 1 hr (1, 5, 15, 30 and 60 min). Cells were fixed in 4% (w/v) paraformaldehyde for 20 min, permeabilized in 0.1% (v/v) saponin for 30 min, and incubated with streptavidin-AF488 for 1 hr and, after washing, mounted with DAPI-containing media and examined by confocal microscopy. (B) P2+G (200 μg/ml) was first incubated with streptavidin-AF488 (50 μg/ml) for 30 min on ice to generate conjugates which were then incubated for 1 hr with DC on coverslips without prior fixation or permeabilization. After washing, cells were fixed and mounted with DAPI-containing media. Section images were captured (0.36 μm). Scale bars, 20 μm.

FIGS. 19A-B shows micrographs of concentration-dependent dendritic cell (DC) penetration by P2+G (P2M6) and its streptavidin conjugates. (A) DC on coverslips were incubated for 1 hr on ice with P2M6 at 10, 25, 50, 100 and 200 μg/ml. Cells were fixed in 4% (w/v) paraformaldehyde for 20 min and permeabilized in 0.1% (v/v) saponin for 30 min to incubate with streptavidin-AF488 (50 μg/ml). After washing, cells were mounted with DAPI-containing media and examined by confocal microscopy. (B) different concentrations of P2+G (10, 25, 50, 100 and 200 μg/ml) were incubated with streptavidin-AF488 for 30 min on ice and the conjugates were then incubated with DC. After washing, cells were fixed and, without permeabilization, mounted with DAPI-containing media and analyzed by confocal microscopy. Section images were captured (0.36 μm). Scale bar, 20 μm.

FIGS. 20A-B shows sequences of P2+G, P2+G(F/I), P2+G(F/L), P2+G(G/A) and P2+G(G/P) mutants thereof, and their alarmin activities. (A) With P2+G as a template, 4 more mutant peptides were synthesized either by changing the 4 phenylalanine residues to isoleucine (P2+G(F/I)) or leucine (P2+G(F/L)) residues, or by changing 6 of its 25 glycine residues into alanine (P2+G(G/A)) or proline (P2+G(G/P)) residues. (B) These four new peptides were used to stimulate PBMC for 24 hr and TNFα production was measured by ELISA. P2+G and P2R/K were used as positive and negative controls, respectively.

FIGS. 21A-B shows the alarmin activities of other known, non-GAR/RGG type of cell-penetrating peptides (CPPs) (Table 1). A) With P2+G as a positive control, P2F/R as an intermediate control, and P2R/K as a negative control, seven known CPPs which lack the GAR/RGG sequence were used to stimulate PBMC. After 24 hr, TNFα production was measured in the media. B) With P2+G as a positive control, CPP4, its tandem dimer (2×CPP4), and 10 mutants were used to stimulate PBMC. After 24 hr, TNFα production was measured by ELISA. Experiments were performed in triplicates. Data were analysed using one-way ANOVA and presented as mean±SD. * p<0.05, ** p<0.01, *** p<0.001.

FIGS. 22A-C shows flow cytometry data of cell-penetrating peptide (CPP) activities of peptide P2+G and P2+G(F/I), P2+G(F/L), P2+G(G/A) and P2+G(G/P) mutants thereof. (A) PBMC were incubated for 1 hr at 4° C. with each of the four P2+G mutant peptides. The mutations involved either the phenylalanine or glycine residues in P2+G. The 4 phenylalanine residues in P2+G were changed either to isoleucine (P2+G(F/I)) or leucine (P2+G(F/L)). Six of the 25 glycine residues in P2+G were changed to either alanine (P2+G(G/A)) or proline (P2+G(G/P)) residues. These peptides (200 μg/ml) were incubated with PBMC for 1 hr at 4° C. Surface-bound and intracellular peptides were detected with streptavidin-AF488. As controls, PBMC were incubated with P2+G, P2F/R, or P2R/K. Cells were analysed by flow cytometry. The vertical bars were used to indicate the surface (dark vertical line) and intracellular (light vertical line) fluorescence intensity obtained with P2+G which were used as references for those of other peptides. (B) Based on the flow cytometry results in A, mean fluorescence indices (MFI) were calculated and compared. (C) PBMC were also incubated for 1 hr at 37° C. with the peptides and similarly examined by flow cytometry. Only MFI data are shown for these experiments.

FIG. 23 shows P2+G-induced dendritic cell (DC) maturation. DCs were cultured from monocytes which exhibited the typical surface phenotype of CD14^{lo/-} and CD1a^{hi}. DC were cultured for 48 hr in the presence of LPS (0.5 μg/ml), P2+G (200 μg/ml) or, as a control, PBS. Cells were harvested and surface-stained for CD40, CD80, CD83, CD86 and MHC class II (MHC II). Cells were analysed by flow cytometry (open histograms). As controls for these antibodies, corresponding isotype-matched mouse IgG were used to stain the cells (filled histograms). The vertical bars indicate the peak fluorescence index of MHC II and co-stimulatory molecules expressed on unstimulated DC (control).

FIG. 24 shows loading of dendritic cells (DCs) with a P2+G-fused peptide antigen enables DC to activate autologous CD4 and CD8 T cells. DC were cultured from monocytes and then incubated for 24 hr with a 30-AA peptide antigen IPA1E2 (SEQ ID NO: 57), the P2+G peptide, or a IPA1E2-P2+G fusion peptide (SEQ ID NO: 58) without additional adjuvant stimulation. These antigen-loaded DC were then co-cultured with lymphocytes from the same blood donor which were labelled with CellTrace Violet. The DC:T cell ratio was 1:5. After 2 weeks, the co-cultured cells were stained with anti-CD14 (monocytes, BV711), anti-CD3 (T cells, PerCP-Cy5-5), and anti-CD19 (B cells, Pacific blue) antibodies, and were also stained with the Zombie NIR Cell Viability reagent (APC-Cy7, BioLegend). CD4⁺ T cells, CD8⁺ T cells, and CD19⁺ B cells were separately analysed by flow cytometry to measure cellular levels of CellTrace Violet reduction due to proliferation. The percentage of proliferated cells were presented. Data were from three independent blood donors and analysed by student t test, *p<0.05, **p<0.01.

FIGS. 25A-B shows experiments examining the cytolytic activities of NCL-P2, P2 mutant peptides, and seven known non-GAR/RGG cell-penetrating peptides (CPPs). A) Buffy coat (2.5 ml) was washed first with 7.5 ml of 150 mM NaCl and then PBS (pH 7.4) by centrifugation for 5 min at 500×g. The cell pellet was resuspended in 7.5 ml of PBS. In 96-well V-bottom plates, peptides (2 mg/ml) were added in triplicates at 10 μl/well. As controls, wells were added with 10 μl of PBS or 20% (v/v) Triton X-100. Cells were diluted 50-fold in PBS and then added to each well at 190 μl/well. After incubation for 1 hr at 37° C., plates were centrifuged for 5 min at 500×g. The supernatants were transferred to 96-well flat-bottom plates at 100 μl/well and measured at OD₄₀₅. Hemolysis in each well was normalized to the average reading of the Triton X-100 control wells which was taken as 100. B) P2+G was first diluted in PBS from 4 mg/ml to 2, 1, 0.5, 0.25, 0.125, and 0.0625 mg/ml. In triplicates, the diluted P2+G was added into 96-well V-bottom plates at 10 μl/well. As controls, wells were added with 10 μl of PBS or 20% (v/v) Triton X-100. Diluted cells were added to each well at 190 μl/well. After incubation for 1 hr at 37° C. and centrifugation, the supernatants were measured at OD₄₀₅, normalized to the average absorbance of wells containing Triton, and presented as percentage hemolysis. Experiments were performed in triplicates. Data were analysed using one-way ANOVA and presented as mean±SD. * p<0.05, ** p<0.01, *** p<0.001.

FIG. 26 shows a schematic diagram of one expected application of P2+G and its related peptides in vaccine development based on the Examples herein. Here P2+G is used as an example. P2+G can activate TLR2 and probably TLR4 [Wu, S., et al., Cell Death Dis 12: 477 (2021)].

### DETAILED DESCRIPTION OF THE INVENTION

Bibliographic references mentioned in the present specification are for convenience listed at the end of the examples. The whole content of such bibliographic references is herein incorporated by reference.

The present invention is based, in part, on the development of a peptide and variants thereof that have alarmin and/or cell penetrating activity. The cell penetrating activity not only improves presentation of a fused antigen to the immune system, but presents opportunities to transport other molecules (cargo molecules) such as nascent protein strands, nucleic acids or small molecules into cells. As described herein, peptides of the invention have adjuvant activity and present advantages as components of vaccines.

### Definitions

Certain terms employed in the specification, examples and appended claims are collected here for convenience.

As used in the specification and the appended claims, the singular forms “a,” “an” and “the” include plural referents unless the context clearly dictates otherwise.

As used herein, ranges can be expressed as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that each unit between two particular units are also disclosed. For example, if 10 and 15 are disclosed, then 11, 12, 13, and 14 are also disclosed.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

As used herein, the terms “polypeptide”, “peptide” or “protein” refer to one or more chains of amino acids, wherein each chain comprises amino acids covalently linked by peptide bonds, and wherein said polypeptide or peptide can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The term “variant”, or “mutant” as used herein, refers to an amino acid sequence that is altered by one or more amino acids, but retains alarmin and/or cell-penetrating activity. The variant may have amino acid deletions or insertions, or both. Guidance in determining which amino acid residues may be substituted, inserted, or deleted without abolishing biological or immunological activity may be found using computer programs well known in the art, for example, DNASTAR® software (DNASTAR, Inc. Madison, Wis., USA). For example, the addition of a ‘G’ amino acid residue into NCL-P2 peptide (NCL-P2+G) increased adjuvant activity three-fold compared to NCL-P2 peptide. The addition of a further two ‘G’ residues did not further improve NCL-P2+G peptide, although the variant retained adjuvant activity. A “polypeptide”, “peptide” or “protein” can comprise one (termed “a monomer”) or a plurality (termed “a multimer”) of amino acid chains.

As used herein, the term ‘fusion polypeptide’ is to be understood as a peptide of the invention conjugated or joined to an entity such as a peptide antigen or cargo molecule. Such fusion could be generated through recombinant DNA methods, peptide synthesis, or chemical conjugation. A peptide linker may be used in some circumstances where spacing between the peptide and antigen or cargo molecule improves effectiveness of the fusion polypeptide. Moreover, “fusion” refers to the joining of a peptide of the invention to an antigen peptide of interest in-frame such that the peptide and antigen or cargo molecule are linked to form a fusion, wherein the fusion does not disrupt the formation or function of the peptide (e.g., its ability to act as an adjuvant and/or penetrate cells) or the attached antigen or cargo molecule. In certain embodiments, the polypeptide/antigen or cargo molecule is fused to the carboxy-terminus of the peptide of the invention. For example, a fusion polypeptide according to any aspect of the present invention may comprise an NCL-P2+G peptide fused to the peptide antigen IPA1E2 as shown in Example 14.

The term “adjuvant”, in the context of the invention is used interchangeably with the term “alarmin” and refers to an immunological adjuvant. By this, an adjuvant is a peptide compound that is able to enhance or facilitate the immune system's response to an attached antigen in question, thereby inducing an immune response or series of immune responses in the subject. For example, DC exposed to the NCL-P2+G peptide fused to the antigen IPA1E2 caused significantly increased T cell proliferation, as shown in Example 14.

As used herein, the term ‘cargo molecule’ is intended to include molecules such as nascent protein strands, nucleic acids or small molecules that can be fused to the peptide adjuvant and be transported into cells by virtue of cell penetrating activity of said peptide adjuvant of the invention.

As used herein, the term “carrier” or “carrier function” refers to, for example, peptides of the invention which are generally fused to cargo molecules and capable of carrying them to and/or into a cell. Preferably such carrier peptides have cell-penetrating activity. Examples include but are not limited to NCL-P2F/Y, NCL-P2F/W and NCL-P2F/R.

The term “active fragment” refers to a portion of a protein that retains some or all of the activity or function (e.g., biological activity or function, such as alarmin/adjuvant activity) of the full-length peptide adjuvant, such as, e.g., the ability to stimulate the immune system and/or penetrate cells. The active fragment can be any size, provided that the fragment retains, e.g., the ability to stimulate the immune system.

The terms “variant” and “mutant are used interchangeably in the context of the invention to refer to a peptide that may be modified by varying the amino acid sequence to comprise one or more naturally-occurring and/or non-naturally-occurring amino acids, provided that the peptide analogue is capable of acting as an adjuvant and/or as a cell-penetrating peptide. For example, these terms encompass a GAR/RGG-rich peptide comprising one or more conservative amino acid changes. Advantageously, the variant/mutant comprises an insertion of one or more ‘G’ residues to complete a triplet, such as “RGRGG” to “RGGRGG”, or “RGGFRGG” to “RGGFGGRGG”. Mutating the GAR/RGG-rich peptide by substituting certain amino acids can improve or diminish the peptide's adjuvant and/or cell-penetrating activity. The term “variant”/“mutant” also encompasses a peptide comprising, for example, one or more D-amino acids. Such a variant has the characteristic of, for example, protease resistance. Variants also include peptidomimetics, e.g., in which one or more peptide bonds have been modified.

As used herein, the term “nucleic acid” refers to a polymer comprising multiple nucleotide monomers (e.g., ribonucleotide monomers or deoxyribonucleotide monomers). “Nucleic acid” includes, for example, genomic DNA, cDNA, RNA, and DNA-RNA hybrid molecules. Nucleic acid molecules can be naturally occurring, recombinant, or synthetic.

As will be appreciated by those of skill in the art, in certain embodiments, the nucleic acid further comprises a plasmid sequence. The plasmid sequence can include, for example, one or more sequences of a promoter sequence, a selection marker sequence, or a locus-targeting sequence. Methods of introducing nucleic acid compositions into cells are well known in the art.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

### EXAMPLES

A person skilled in the art will appreciate that the present invention may be practiced without undue experimentation according to the methods given herein. The methods, techniques and chemicals are as described in the references given or from protocols in standard biotechnology and molecular biology textbooks. Standard molecular biology techniques known in the art and not specifically described were generally followed as described in Sambrook and Russel, Molecular Cloning: A Laboratory Manual, Cold Springs Harbor Laboratory, New York (2001).

### Example 1

### Materials and Methods

### Antibodies and Reagents

Rabbit polyclonal antibodies against NCL (ab22758) and HMGB1 (ab67281) were obtained from Abcam (Cambridge, UK). A mouse monoclonal anti-NCL antibody was purchased from Santa Cruz. Lipopolysaccharide (LPS) and mouse IgG1 (M9269) were purchased from Sigma-Aldrich. Recombinant human TLR2-10×His (R&D Systems, Mineapolis, Minn.) was obtained from R&D Systems. The anti-HA-agarose resins and streptavidin Alexa Fluor 488 were obtained from ThermoFisher Scientific (Walthem, Mass.). A TLR4-blocking mouse antibody (Mabg-htlr4), a TLR5-blocking human antibody (Maba-htlr5), an interleukin (IL)-1□-blocking mouse antibody, lipoteichoic acid (LTA, tlr1-slta), flagellin stfla), and poly I:C (tlrl-picw), were from InvivoGen (San Diego, Calif.). Peptides were synthesized with or without N-terminal biotin-Ahx by ChemPeptide Ltd (Shanghai, China). Mouse antibodies for CD14 (BV711), CD3 (PerCP-Cy5-5), CD19 (Pacific blue), CD40 (BV785), and the Zombie NIR Cell Viability reagent (APC-Cy7) were obtained from Biolegend (San Diego, Calif.). Antibodies for CD1a (PE, #145-040), CD86 (FITC, #307-040) and MHC II (FITC, #131-040) were obtained from Ancell Co. (Bayport, Minn.). Antibody for CD14 (PE, #MA1-80587) was obtained from Invitrogen). Antibodies for CD80 (PE, #557227) and CD83 (PE, #556855) were purchased from BD.

### Protein Purification

The nuclear extract (T×NE) was isolated from HeLa cells as previously reported [Chen, J., et al., J Biol Chem 293: 2358-2369 (2018)] and used to affinity-purify nuclear proteins. Briefly, antibodies (60 μg) specific for NCL, HMGB1 or non-immune mouse IgG1, were first bound to 600 μl of protein G-Sepharose beads (GE Health) overnight and the beads were, after washing, incubated for 30 min with 0.2 M dimethyl pimelimidate (DMP) in PBS containing triethanolamine, pH 8-9. The resins were washed three times in the PBS-triethanolamine buffer and blocked in PBS containing ethanolamine (50 mM). The resins were first eluted using 0.1 M glycine (pH 2.5) and then equilibrated in TBS (50 mM Tris, pH 7.4 and 150 mM NaCl). The resins were incubated overnight with T×NE and, after washing with 50 ml of wash buffer (0.25 M sucrose, 10 mM Tris, 3.3 mM CaCl₂), 0.1% (v/v) Tween 20), eluted using 0.1 M glycine (pH 2.5) collecting 10×0.3 ml fractions. Protein concentrations were determined based on OD₂₈₀ reading and protein-containing fractions (usually fractions 1-3) were combined. Endotoxin contamination was tested for using an LAL Endotoxin Assay (Genscript Piscataway, N.J.).

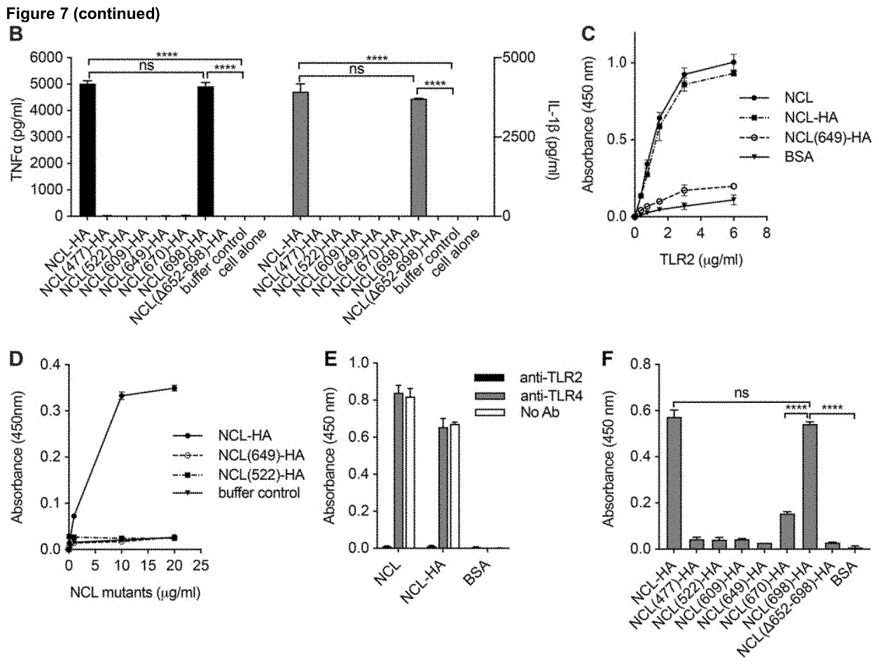

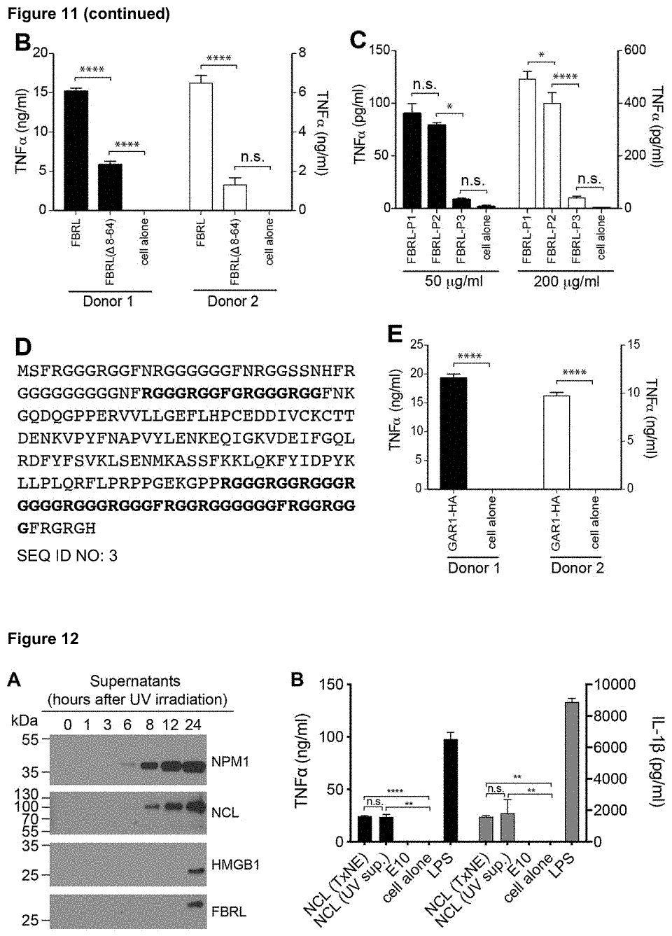

To purify recombinant nuclear proteins, three master expression vectors were generated using the pcDNA3.1 vector (Invitrogen, Waltham, Mass.) that encode full-length NCL, FBRL and GAR1, respectively (FIG. 7A, FIG. 11). Vectors that express NCL and FBRL deletion mutants were also generated as detailed. All these recombinant nuclear proteins or mutants contain a C-terminal HA tag. After transfection into HEK293T cells using the calcium phosphate method [Cao, W., et al., Blood 107: 2777-2785 (2006)], HEK293T cells were cultured in DMEM containing 10% (v/v) heat-inactivated serum (FBS), 2 mM of L-glutamine and 100 units/ml of penicillin/streptomycin in the presence of 5% CO₂. Transfected cells were harvested after 48 hr and homogenized to separate nuclei from cytoplasm and T×NE was isolated from the nuclei to combine with the cytoplasm [Chen, J., et al., J Biol Chem 293: 2358-2369 (2018)]. This cell lysate was incubated overnight at 4° C. in a column with 0.3 ml of anti-HA-agarose (ThermoFisher Scientific). After washing with 50 ml of a wash buffer (0.25 M sucrose, 10 mM Tris, pH 7.4, 250 mM NaCl, 3.3 mM CaCl₂), and 0.1% Tween 20), bound proteins were eluted using 3.5 M MgCl₂ to collect 10×0.3 ml fractions. SDS-PAGE was used to detect the eluted proteins and the fractions were combined and dialyzed in PBS. Protein concentrations were then determined based on OD₂₈₀ reading.

### SDS-PAGE and Western Blotting

Protein samples were diluted to 10 mM with dithiothreitol and boiled for 10 min at 100° C. before separation on 12.5% (w/v) SDS-PAGE gels. Gels were stained with Coomassie blue to view proteins. For Western blotting, the gels were electro-blotted onto PVDF membranes which were first blocked for 1 hr with 5% (w/v) non-fat milk in TBS-T (50 mM Tris pH 7.4, 150 mM NaCl and 0.1% (v/v) Tween 20) and then incubated overnight at 4° C. with specific antibodies.

After washing, the membranes were exposed to horseradish peroxidase (HRP)-conjugated secondary antibodies for 1 hr and developed using the Pierce SuperSignal West Pico chemiluminescent substrate (ThermoFisher Scientific).

### Cell Isolation and Culturing

Buffy coat fractions were obtained from healthy blood donors at the Singapore Health Sciences Authority, with Institutional ethics approval, and PBMC were isolated using Ficoll-Paque (GE Healthcare). To isolate monocytes, PBMC were re-suspended to 1×10⁷ cells/ml in the RPMI medium contained 5% (v/v) BCS and incubated for 1 hr in T75 flasks (20 ml/flask). Monocytes that adhered were harvested. To culture macrophages and DC [Cao, W., et al., Blood 107: 2777-2785, (2006), incorporated herein by reference], monocytes were resuspended to 1×10⁶ cells/ml and cultured in 6-well plates (2 ml/well). Macrophages were cultured by adding M-CSF to 20 ng/ml and DC were cultured with 20 ng/ml GM-CSF and 40 ng/ml IL-4. M-CSF, GM-CSF and IL-4 were obtained from R&D Systems (Mineapolis, Minn.). Cells were cultured for 6 days with half of the media being replenished every two days.

### Cell Activation

Purified proteins in PBS (30 μg/ml) were coated in triplicates in 96-well plates (50 μl/well) for 12 hr and PBMC (3×10⁶ cells/ml), monocytes (1×10⁶ cells/ml), macrophages (0.5×10⁶ cells/ml) or DC (0.5×10⁶ cells/ml) were re-suspended in macrophage serum-free medium containing penicillin and streptomycin and cultured for 24 hr in these plates at 100 μl/well. Where TLR ligands were used to stimulate these cells, they were added to the media: LPS (500 ng/ml for DC and macrophages and 10 ng/ml for PMBC and monocytes, InvivoGen), flagellin (1 μg/ml, InvivoGen), lipoteichic acid (LTA, 10 μg/ml). Cell activation was determined by measuring TNFα and IL-1β in the culture media using ELISA kits (Invitrogen).

In some experiments, cells were pre-treated with the MyD88 inhibitor st-2825 (MedChemExpress) or the Caspase-1 inhibitor Ac-YVAD (InvivoGen) for 1 hr before stimulation with TLR ligands or the purified nuclear proteins. In some other experiments, cells were pre-incubated for 1 hr with anti-TLR2, TLR4 and TLR5 antibodies (InvivoGen) before stimulation. The optimal st-2825 and Ac-YVAD concentrations were determined based on both their effects on cell viability and LPS-induced cytokine production. Cell viability was determined using the CELLTITER 96® AQueous One Solution Cell Proliferation (MTS) Assay (Promega).

In some experiments, to detect surface proteins on cultured DC, cells were harvested at day 6 and re-suspended at 1×10⁵/ml in macrophage serum free medium (Thermo Fisher Scientific, cat #12065074). Cells were incubated for 1 hr on ice with fluorescent antibodies specific for CD14 (PE), CD1a (PE), or isotype-matched IgG. Cells were washed and analysed by flow cytometry. The harvested DC were also resuspended in the medium at 5×10⁴/ml and cultured for 48 hr with LPS (0.5 μg/ml), P2M6 (200 μg/ml) or, as a control, PBS. Cells were then incubated with fluorescently tagged antibodies specific for MHC class II, CD40, CD80, CD83, CD86, and corresponding isotype controls. Cells were analysed by flow cytometry.

### Confocal Microscopy

DC were harvested and cultured overnight on glass coverslips. The cells were first incubated for 1, 5, 15, 30 or 60 min with P2M6 (200 μg/ml) at 4° C. and then fixed in 4% (w/v) paraformaldehyde (PFA) for 20 min. Cells were permeabilized for 30 min in 0.1% (w/v) saponin and then incubated for 1 hr with streptavidin-AF488 (50 μg/ml). Cells were then mounted for imaging analysis. Alternatively, P2M6 was pre-incubated for 1 hr on ice with streptavidin-AF488 at 50 μg/ml and the peptide-streptavidin complexes were at 1/10 dilution incubated with DC for 1, 5, 15, 30 or 60 min at 4° C. and the cells were, after washing, directly mounted without fixation or permeabilization.

In another experiment set up, DC (2×10⁵/ml) were incubated for 1 hr at 4° C. with P2M6 at different concentrations (10, 25, 50, 100, or 200 μg/ml). Cells were fixed and permeabilized to incubate for 1 hr with streptavidin-AF488 (50 μg/ml). Cells were washed and mounted for imaging analysis. Alternatively, Different concentrations of P2M6 (100, 250, 500, 1000 or 2000 g/ml) were pre-incubated for 1 hr on ice with streptavidin-AF488 (500 μg/ml). The preformed complexes were at 1/10 dilutions incubated with DC for 1 hr at 4° C. The cells were, after washing, directly mounted without fixation or permeabilization.

All cells were mounted using the VectaShield mounting medium containing DAPI (Vector Laboratories). Cells were analyzed using the FluoView FV3000 confocal microscope equipped with a 100× oil objective (aperture 1.45) and Cool/SNAP HQ2 image acquisition camera (Olympus). Images were captured with the FV-ASW 1.6b software and analyzed using the Imaris software (Bitplane AG).

### Hemolytic Assay

Buffy coats were used as a source of red blood cells (RBC). Buffy coat (2 ml) was washed first in 10 ml of 150 mM NaCl and then washed twice in PBS (pH 7.4) by centrifugation for 5 min at 500 g. The cell pellets were resuspended in 10 ml of PBS as RBC stocks. The different peptides were diluted in PBS (100 μg/ml) and, in triplicates, the peptides were added to V-bottom 96-well plates at 10 μl/well. As controls, the same volumes of PBS or 20% (v/v) Triton X-100 were added. RBC were diluted 50 times in PBS and added to the plates at 190 μl/well. After incubation for 1 hr at 37° C., the plates were centrifuged for 5 min at 500 g. The supernatants (100 μl/well) were transferred to flat bottom plates and absorbance was measured at OD405. Data were normalized to the average OD405 readings obtained with 1% (v/v) Triton X-100 and presented as percentage hemolysis.

### TLR and NF-κB-Luciferase Assay

TLR-mediated NF-κB activation was determined using a Dual Luciferase Reporter Assay (Promega), in which two luciferase reporter plasmids were used. One plasmid expresses the firefly luciferase under the regulation of inducible NF-κB promoter and the other plasmid expresses the Renilla luciferase under a constitutively active CMV promoter [Zhang, H., et al., FEBS Lett 532: 171-176 (2002)]. Besides these luciferase vectors, cells were co-transfected with vectors coding for human TLRs or, in the case of TLR4, co-transfected with CD14 and MD2 [according to Zhang, H., et al., J. FEBS Lett 532: 171-176 (2002), incorporated herein by reference]. Transfection was performed using the TurboFect Transfection Reagent (Thermo Fisher Scientific). After 24 hr, cells were harvested and cultured for 24 hr in 96-well plates coated with the purified proteins or, as controls, cultured in blank plates but stimulated with TLR ligands. Cells were lysed to measure both firefly and Renilla luciferase activities and, in each sample, the firefly activity was normalized to the Renilla luciferase activity and expressed as relative NF-κB activation.

### TLR2 Binding Assay

96-well ELISA plates were coated overnight at 4° C. with purified nuclear proteins in PBS at 100 μl/well (10 μg/ml) in duplicates. Plates were washed in PBS containing 0.05% (v/v) Tween 20 three times and blocked for 1 hr with PBS containing 1% (w/v) bovine serum albumin (PBS-BSA). TLR2-10×His was serially diluted in PBS-BSA to 0.375-6 μg/ml (R&D Systems) and incubated with the coated plates overnight at 4° C. Bound TLR2-10×His was detected by first incubating for 1 hr with mouse anti-His antibody (Sigma) and then incubated for 30 min with HRP-conjugated secondary antibody (DAKO). Plates were developed with the 3, 3′, 5, 5′-Tetramethylbenzidine (TMB) substrate solution (Thermo Fisher Scientific) and stopped by adding 50 μl of 2 N H₂SO₄. Absorbance was measured at 450 nm.

### Peptide Binding to PBMC

PBMC re-suspended in 100 μl macrophage serum free media (3×10⁶/ml) were incubated with different peptides (200 μg/ml). PBMC (100 μl) were incubated with the peptides for 1 hr at 37° C. or 4° C. Cells were washed twice in 2% FBS/PBS and incubated with streptavidin-AF488 and Zombie (NIR) Fixable viability stain-APC-Cy7 for 30 min at 4° C. Cells were then fixed with 1% PFA for 30 min at room temperature and analysed using the Fortessa analyser (BD). In some experiments, PBMC were, after incubation with peptides, incubated with Zombie (NIR) Fixable viability stain (APC-Cy7) for 30 min at 4° C. Cells were then fixed and permeabilized with BD CYTOFIX/CYTOPERM™ Kit for 20 min at 4° C., and then incubated with streptavidin-AF488 for 30 min at 4° C. In some experiments, PBMC were, after incubation with the peptides, stained with fluorescent mouse antibodies specific for monocytes (CD14/BV711), T cells (CD3/PerCP-Cy5-5) and B cells (CD19/Pacific blue). Cells were then stained with Zombie (NIR) Fixable viability stain-APC-Cy7 and streptavin-AF488, with or without membrane permeabilization. In these experiments, monocytes, T cells and B cells were separately gated to detect surface peptide binding and intracellular peptide penetration.

### Example 2

Nucleolin is a Potent Alarmin that Activates PBMC, Monocytes, Macrophages and Dendritic Cells

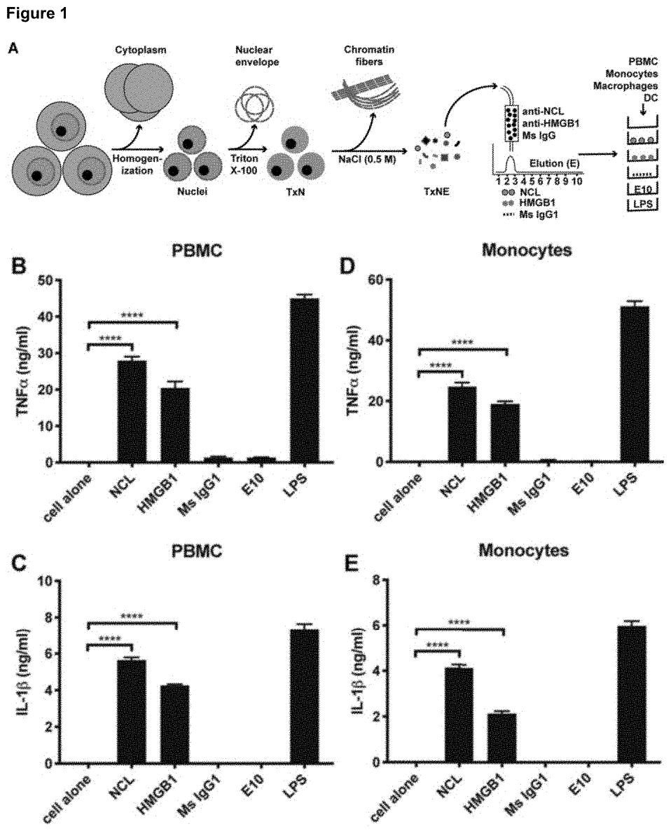

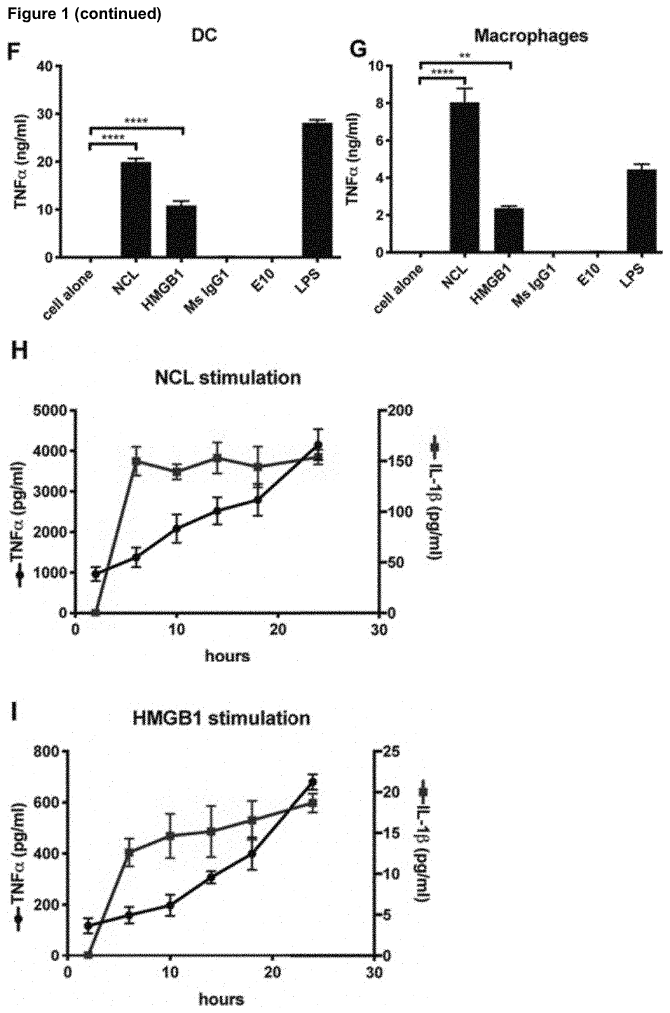

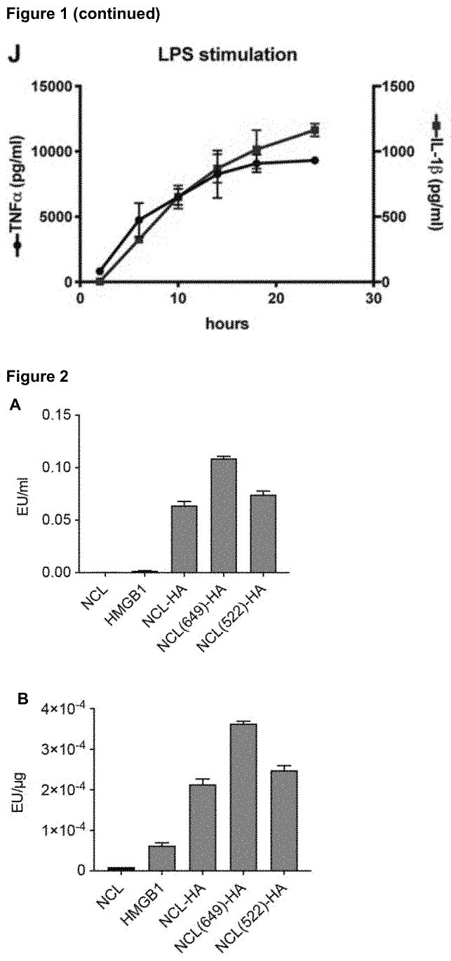

Nucleolin was affinity-purified from the lipid-depleted nuclear extract T×NE to stimulate peripheral blood mononuclear cells (PBMC) [Chen, J., et al., J Biol Chem 293: 2358-2369 (2018), incorporated herein by reference)] (FIG. 1A). HMGB1 was purified as an alarmin control (FIG. 1A). To prepare negative controls, a non-immune mouse IgG1 column was also generated. These affinity resins were incubated with T×NE and bound proteins were eluted after washing. Ten fractions were eluted from each column and the protein-free fraction 10 from multiple elution were combined to use as a second negative control. Endotoxin contamination was monitored using the Limulus amoebocyte lysate endotoxin assay (GenScript, Piscataway, N.J.) (e.g. FIG. 2).

NCL was coated on the plates to stimulate PBMC which consistently induced TNFα and IL-1β production (FIG. 1B, C). These cytokines were similarly induced from monocytes (FIG. 1D, E). As controls, neither elution from the non-immune IgG column nor the combined fractions 10 induced these cytokines but HMGB1 did (FIG. 1B-E). Both NCL and HMGB1 also induced cytokine production from dendritic cells (DC) and macrophages (FIG. 1F, G). Overall, NCL induced more cytokines than HMGB1 which is known to activate TLR2, TLR4 and TLR5 [Sims, et al., Annu Rev Immunol 28: 367-388 (2010); Li, J. et al., Mol Med 9: 37-45 (2003)]. However, NCL is distinct from HMGB1 by sequence.

### Example 3

### Nucleolin Activates TLR2

The two proteins NCL and HMGB1 were compared regarding their kinetics of TNFα and IL-1β induction from PBMC by stimulating these cells with HMGB1, NCL or as a control LPS for up to 24 hr during which TNFα and IL-1β production was measured at 2.5, 5.0, 10, 14, 18 and 24 hr (FIG. 1H-J). NCL and HMGB1 induced IL-1β following similar kinetics which rapidly surged to plateau (FIG. 1H, I). TNFα was also similarly induced by the two proteins exhibiting linear early increase but a noticeable late surge (FIG. 1H, I). The late surge in TNFα induction is most likely due to secondary and autocrine PBMC stimulation by the IL-1β they produce (FIG. 4). LPS-stimulated PBMC exhibited neither early IL-1β surge nor late TNFα surge (FIG. 1J). The similar cytokine production induced by NCL and HMGB1 suggests they activate similar receptors which, for HMGB1, are known to be TLRs [Sims, G. P., et al., Annu Rev Immunol 28: 367-388 (2010); Li, J. et al., Mol Med 9: 37-45 (2003)].

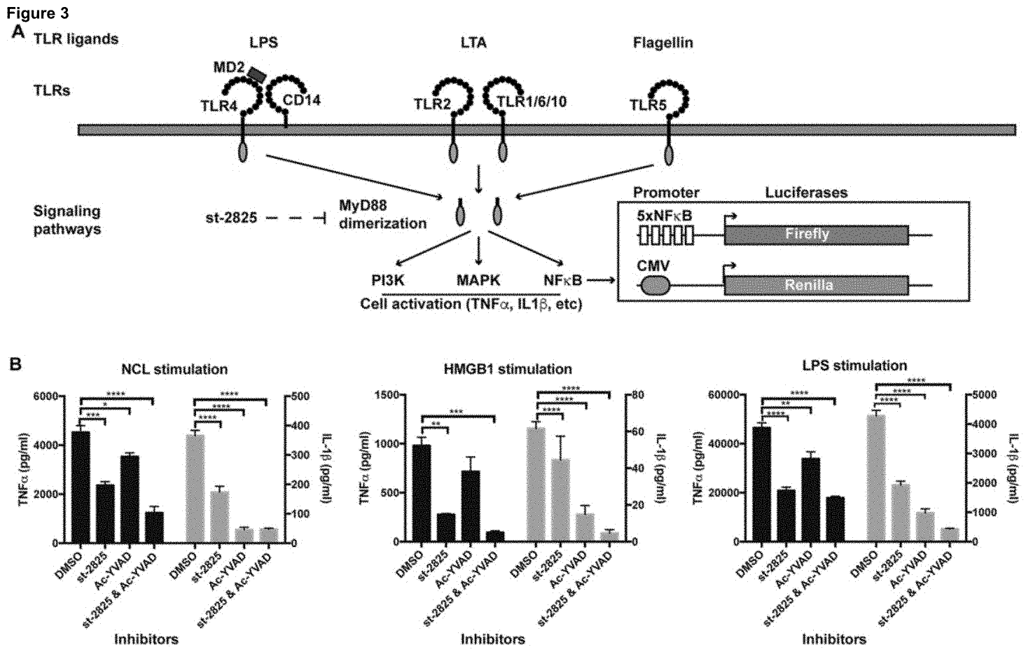

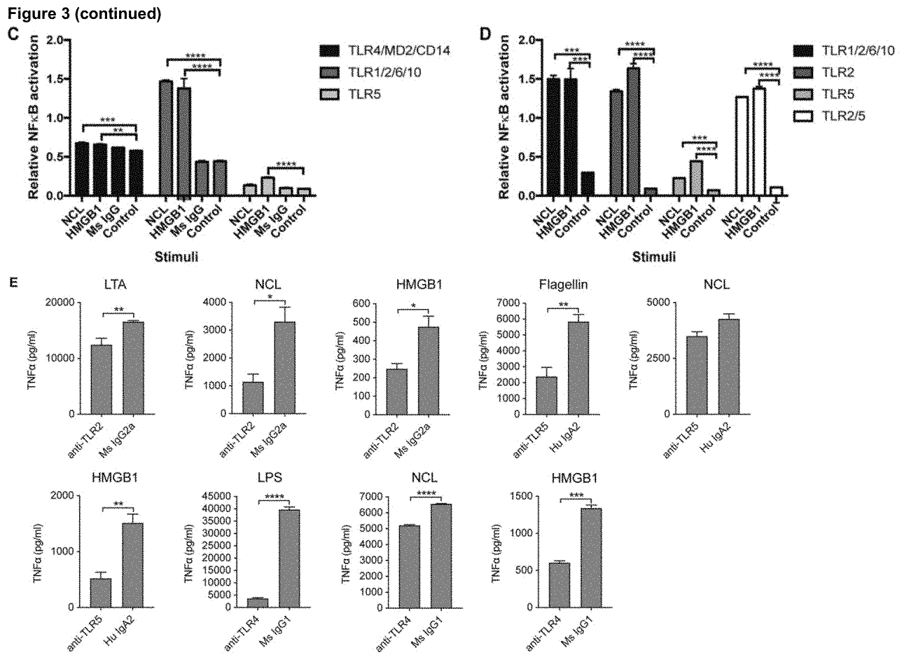

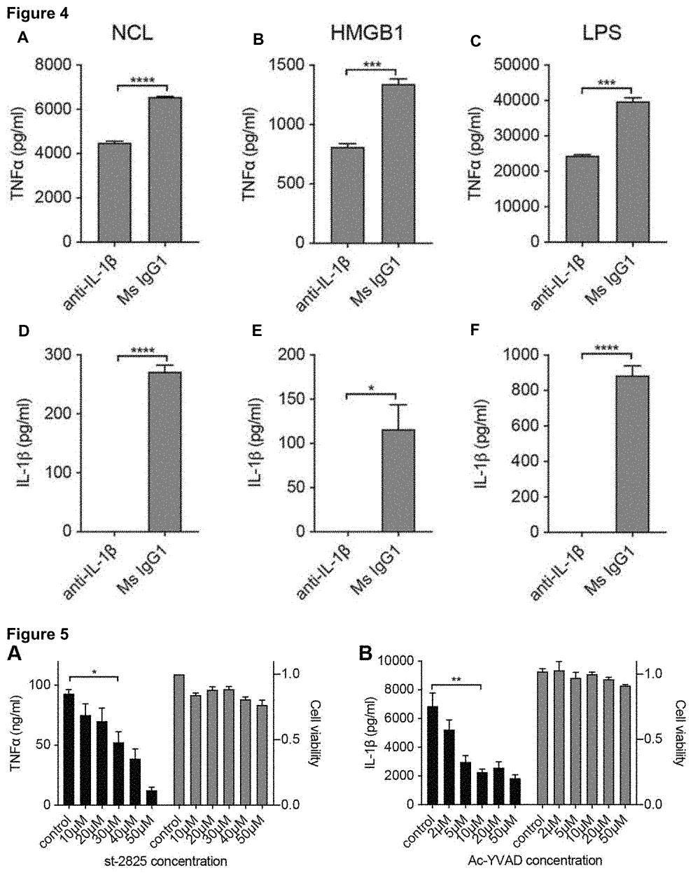

We then examined whether NCL still induces cytokines when MyD88 is inhibited [Kawai, T. and Akira, S., Semin Immunol 19: 24-32 (2007)]. A MyD88 inhibitor st-2825 was used in this experiment (FIG. 3A). Its optimal concentration was, after titration of its cytotoxicity and its inhibition of LPS-induced cytokine production, determined to be 30 μM (FIG. 5). A caspase I inhibitor Ac-YVAD was similarly titrated to 10 μM and used to evaluate the contributions of other alarmin sensing pathways (FIG. 5). st-2825 partially but significantly inhibited NCL induction of TNFα and IL-1β from monocytes and, as expected, it also inhibited HMGB1 and LPS induction of these cytokines (FIG. 3B). Ac-YVAD effectively diminished IL-1β induction by all three stimuli and also partially inhibited TNFα induction (FIG. 3B). The inhibition of TNFα production by Ac-YVAD could be explained by its blocking autocrine monocytes activation through the IL-1β these cells produce (FIG. 4).

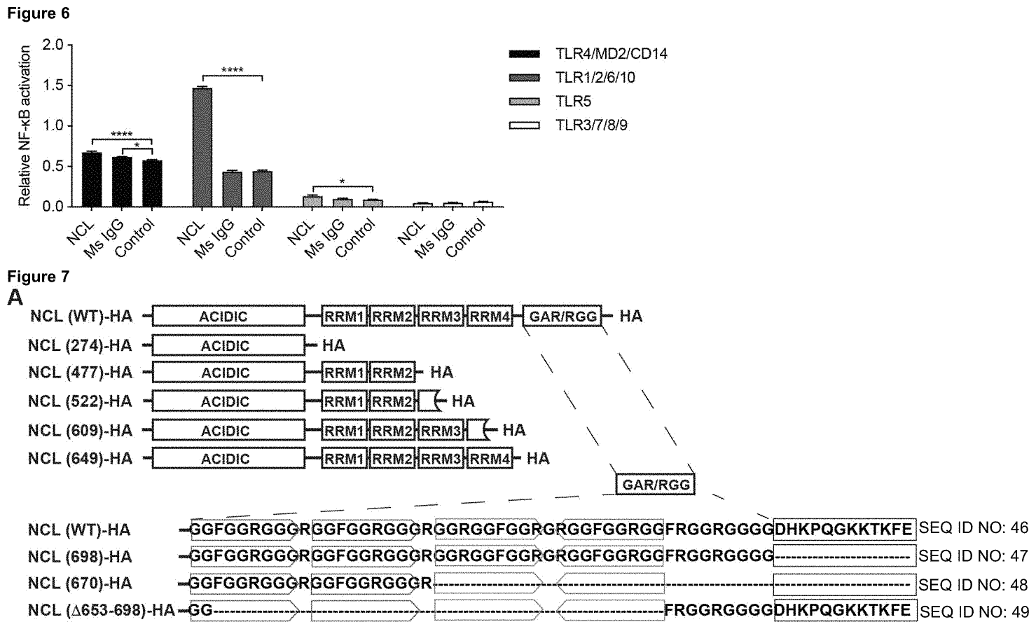

To determine which TLR(s) NCL may activate, a luciferase assay was adopted in which NF-κB-directed luciferase expression vectors were transfected into the human embryonic kidney 293T cells (FIG. 3A) [Zhang, H., et al., FEBS Lett 532: 171-176 (2002), incorporated herein by reference]. TLRs and, where it required, co-receptors were co-transfected in a total of 4 combinations, i.e. TLR2/1/6/10, TLR4/CD14/MD2, TLR5, or TLR3/7/8/9. Two luciferase expression vectors were used: one expresses the firefly luciferase under 5 repeats of the NF-κB gene promoter and the other expresses the Renilla luciferase under the constitutively active CMV promoter (FIG. 3A). Expression of the four intracellular TLRs, TLR3/7/8/9, did not confer detectable response to NCL (FIG. 6). Expression of the TLR4/CD14/MD2 combination caused strong autoactivation as expected [Zhang, H., et al., FEBS Lett 532: 171-176 (2002)] and, on this high background luciferase activity, NCL caused significant albeit marginal additional NF-κB activation (FIG. 6). NCL activation of TLR5 was not consistently observed in the assay, but it strongly activated the TLR2/TLR1/TLR6/TLR10 combination (FIG. 6, FIG. 3C).

Using this assay, NCL and HMGB1 were compared in TLR2, TLR4 and TLR5 activation and both caused prominent activation of the TLR2/TLR1/TLR6/TLR10 combination (FIG. 3C). HMGB1 but not NCL also consistently activated TLR5 (FIG. 3C). Both proteins caused marginal but significant additional TLR4 activation on top of the high TLR4 autoactivation (FIG. 3C). Nonetheless, results support TLR2 as a major receptor for the sensing of NCL and HMGB1. We next determined whether TLR1, TLR6 or TLR10 is required for effective TLR2 response to NCL or HMGB1 and also whether TLR5 would synergize with TLR2 in this function. When TLR2 was expressed without TLR1/TLR6/TLR10 co-expression or co-repressed with TLR5, its response to NCL or HMGB1 was not significantly affected (FIG. 3D).

Therefore, TLR2 is clearly a sensing receptor for NCL as well as HMGB1. We then further analyzed the contribution of TLR2, TLR4 and TLR5 to NCL and HMGB1 recognition in their natural cellular contexts. Monocytes were pre-incubated with antibodies that were known to block each of these TLRs and then stimulated with the respective microbial ligands i.e. lipoteichoic acid (LTA), LPS and flagellin (FIG. 3E). All three antibodies significantly inhibited HMGB1-induced TNFα production from monocytes, which suggest that HMGB1 activates TLR2 as well as TLR5 and TLR4 as reported (FIG. 3E) [Sims, G. P., et al., Annu Rev Immunol 28: 367-388 (2010); Das, N. et al., Cell Rep 17: 1128-1140 (2016)]. Monocyte response to NCL was not significantly affected by the TLR5 antibody and was only marginally inhibited by the TLR4 antibody (FIG. 3E). However, it was strongly inhibited by the TLR2 antibody (FIG. 3E). These results are largely consistent with the luciferase-based conclusion (FIG. 3D). It shows that HMGB1 is more permissively recognized by TLR2, TLR4 and TLR5 but NCL is more selectively recognized by TLR2. NCL is a 710-amino acid protein and it is interesting to identify the specific NCL region that activates TLR2.

### Example 4

### Identification of TLR2-Reactive Regions on NCL

NCL polypeptide (SEQ ID NO: 1) contains 7 domains: a 277-residue N-terminal domain characterized by acidic residues followed by four tandem RNA recognition motifs (RRM1-4) of 375 residues [Maris, C., Dominguez, C. & Allain, F. H., FEBS J 272: 2118-2131 (2005)], an RGG type of glycine and arginine-rich (GAR/RGG) region of 48 residues (SEQ ID NO: 4) [Thandapani, P., et al., Mol Cell 50: 613-623 (2013)], and a short 12-residue C-terminal tail (FIG. 7A). The GAR sequences generally also have RNA-binding properties [Maris, C. Dominguez, C. & Allain, F. H., FEBS J 272: 2118-2131 (2005); Thandapani, P., et al., Mol Cell 50: 613-623 (2013)]. To identify which domain stimulates TLR2 and cytokine production, NCL was expressed in 293 T cells with a C-terminal HA (NCL-HA; GAR/RGG domain set forth in SEQ ID NO: 46) and affinity-purified using an anti-HA antibody column. The recombinant NCL-HA was indistinguishable from endogenous NCL, which was affinity-purified from T×NE using an anti-NCL antibody, in TNFα and IL-1β induction from monocytes (Data not shown). Six NCL-HA mutants were then generated by progressively deleting from the C-terminal end. Only the NCL(698)-HA mutant (GAR/RGG domain set forth in SEQ ID NO: 47) in which 12 amino acids were deleted from the C-terminal end, continued to stimulate monocytes (FIG. 7B). If another 28 residues are deleted beyond the 12 residues into the upstream GAR/RGG, the resultant NCL(670)-HA mutant (GAR/RGG domain set forth in SEQ ID NO: 48) no longer stimulates monocytes (FIG. 7A, B).

The integrity of the 48-residue GAR/RGG region in NCL appeared to be required for TLR2 response to NCL (FIG. 7A). Indeed, internal deletion of 37 residues inside this GAR/RGG region created another inactive NCL(Δ652-698)-HA mutant (GAR/RGG domain set forth in SEQ ID NO: 49) (FIG. 7B). Therefore, the specific TLR2 ligand should reside in the GAR/RGG region.

Next, we investigated whether there is direct binding between TLR2 and NCL and, more specifically, whether TLR2 binds to the GAR/RGG region of NCL. Purified NCL, NCL-HA and the NCL(649)-HA mutant were coated on the plate and then incubated with His-tagged TLR2. BSA was coated as a control. Using an anti-6×His antibody to detect the bound TLR2, it was shown to bind to both NCL and NCL-HA in dose-dependent and saturable manners but there was no binding to the NCL(649)-HA mutant which lacks the GAR/RGG region (FIG. 7C). TLR2 was also coated on the plate and incubated with soluble NCL-HA, NCL(649)-HA and NCL(522)-HA, and bound NCL proteins were detected using an anti-HA antibody. While NCL-HA showed dose-dependent and saturable binding to TLR2, this was not observed with the two NCL mutants which lack the GAR/RGG region (FIG. 7D).

Since NCL stimulation of monocyte surface TLR2 was blocked by a TLR2-specific antibody (FIG. 3E), we examined whether this antibody also blocks NCL binding to TLR2. TLR2 was coated and pre-incubated with the rabbit anti-TLR2 antibody before further incubation with NCL or NCL-HA. As a control, the coated TLR2 was pre-incubated with the rabbit anti-TLR4 antibody (FIG. 3E). Pre-incubation with the anti-TLR2 antibody completely blocked NCL and NCL-HA binding to the coated TLR2, but pre-incubation with the anti-TLR4 antibody showed no inhibition (FIG. 7E). Therefore, TLR2 binds to NCL via the GAR/RGG region and this binding activates its signaling on monocytes that leads to cytokine production.

To ascertain whether TLR2 binds to additional sites on NCL, all 8 available NCL-HA mutants as well as NCL-HA were coated and incubated with TLR2 (FIG. 7F). As expected, TLR2 bound to NCL-HA and NCL(698)-HA and, with the other NCL mutants, TLR2 only showed weak binding to NCL(670)-HA which contains a residual GAR/RGG region (FIG. 7A, F). However, this weak interaction with TLR2 was apparently insufficient for NCL(670)-HA to activate TLR2 and cytokine production (FIG. 7B). Collectively, results show specific TLR2 binding to the GAR/RGG region on NCL which activates monocyte production of cytokines.

### Example 5

### GAR/RGG Peptides can Also be Recognized by TLR2 and Activate Monocytes Through TLR2

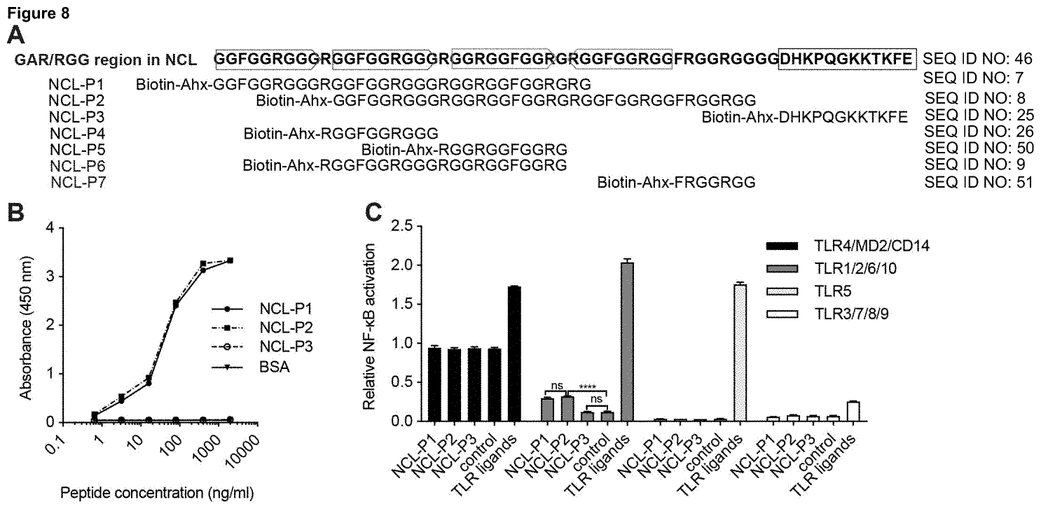

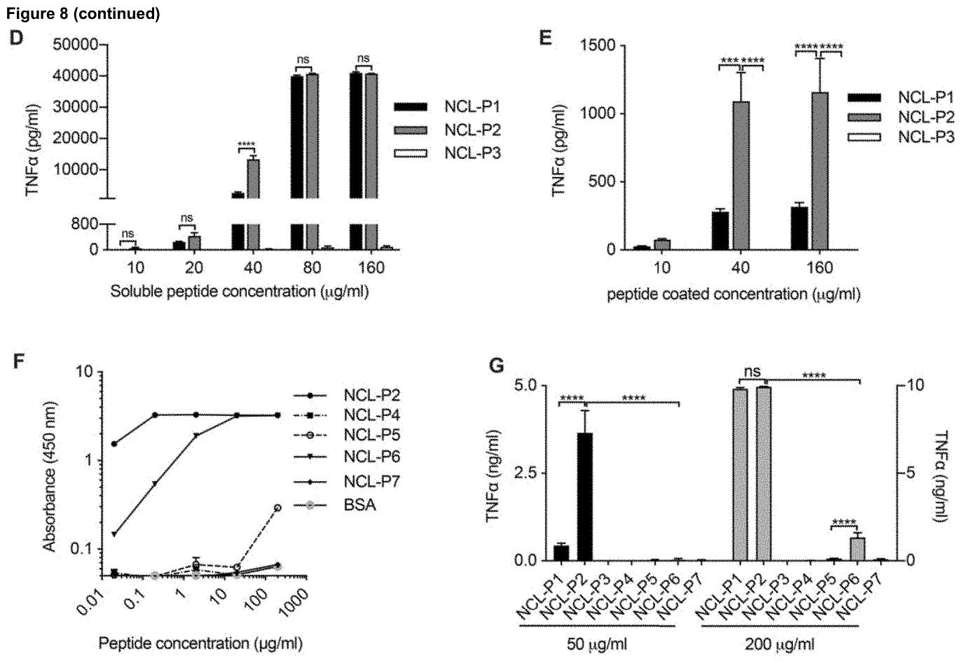

A 48-residue GAR/RGG domain (i.e. from G₆₅₁ to G₆₉₈, SEQ ID NO: 4) within the NCL C-terminal GAR/RGG region (SEQ ID NO: 46) contains four repetitive regions: two head-to-tail repeats (GGFGGRGGGRggfggrgggr; SEQ ID NO: 17) and two tail-to-tail repeats (GGRGGFGGRgRGGFGGRGG; SEQ ID NO: 18), and a non-repetitive C-terminal region (FRGGRGGGG; SEQ ID NO: 19) (FIG. 7A, 8A). To determine whether a specific sequence in this GAR/RGG region is preferentially recognized by TLR2, two overlapping peptides were synthesized with peptide NCL-P1 (32 residues; SEQ ID NO: 7)) covering the N-terminal three repeats and peptide NCL-P2 (36 residues; SEQ ID NO: 8)) covering the C-terminal three repeats (FIG. 8A). A control peptide was also synthesized that covered the C-terminal 12 residues of NCL outside the GAR/RGG domain (SEQ ID NO: 25) (FIG. 8A). NCL-P1 and NCL-P2 were designed to overlap over the middle two repeats. All peptides were synthesized with N-terminal biotin tags.

TLR2 was coated on the plates and incubated with the peptides at increasing concentrations from 0.64 to 1,000 ng/ml. NCL-P1 and NCL-P2 exhibited similar dose-dependent and saturable binding to TLR2 (FIG. 8B). However, NCL-P3 (SEQ ID NO: 25) showed no detectable binding to TLR2. The three peptides were also tested in the NF-κB luciferase assay. NCL-P1 and NCL-P2 caused similar TLR2-mediated NF-κB activation, but this was not observed with NCL-P3 (FIG. 8C). The three peptides were also used to stimulate monocytes by adding to the culture at increasing concentrations from 10 to 160 μg/ml. At all concentrations, NCL-P3 showed no TNFα induction (FIG. 8D). At lower concentrations (10 and 20 μg/ml), NCL-P1 and NCL-P2 also induced little TNFα. However, at higher concentrations (80 and 160 μg/ml), both NCLP1 and NCL-P2 strongly induced TNFα production (FIG. 8D). A difference between NCL-P1 and NCL-P2 was observed at 40 μg/ml when NCL-P2 induced approximately 10-fold more TNFα than NCL-P1 (FIG. 8D). When the two peptides were coated on the plates to stimulate monocytes, NCL-P2 also induced more cytokines than NCL-P1 (FIG. 8E). Therefore, NCL-P2 carries stronger alarmin activity than NCL-P1.

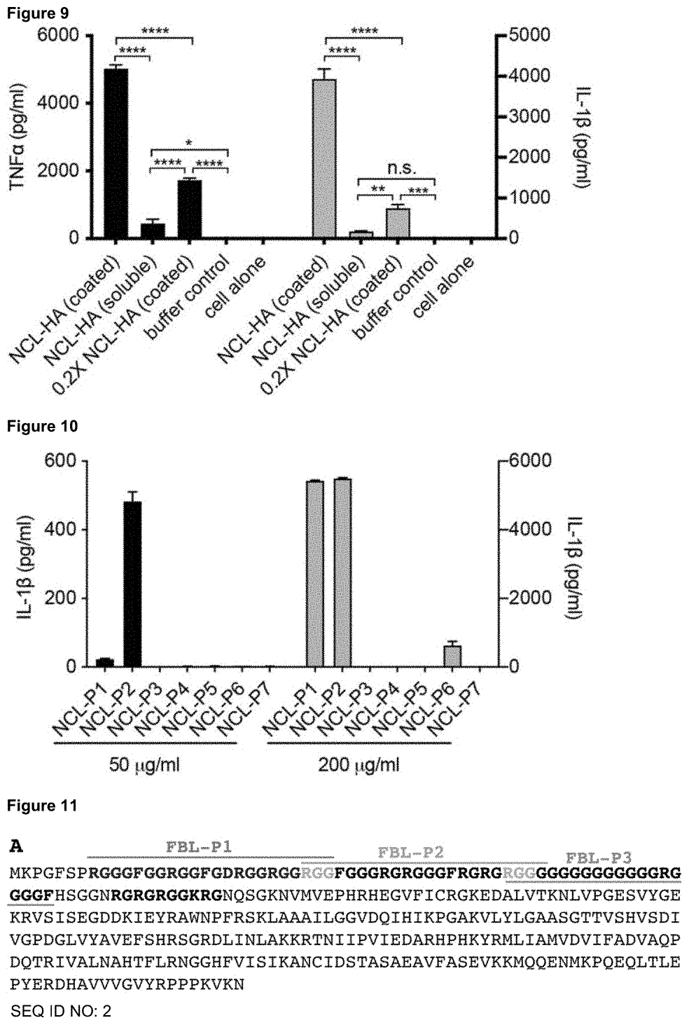

Overall, soluble NCL-P2 and NCL-P1 induced more cytokines than immobilized peptides (FIG. 8D, 8E). In contrast, surface-coated NCL-HA induced more cytokines than soluble NCL-HA (FIG. 9). We suspect that coating a large protein like NCL on the plates creates multivalent GAR/RGG stimulation of TLR2 but coating the short GAR/RGG peptides may hinder TLR2 access to these sequences.

To further understand this novel TLR2 ligand region on NCL, we synthesized one peptide (NCL-P6; SEQ ID NO: 9) corresponding to the overlapping sequences between NCL-P1 and NCL-P2 and two more peptides (NCL-P4; SEQ ID NO: 26 and NCL-P5; SEQ ID NO: 50), each covering half of this common sequence (FIG. 8A). Another short peptide was synthesized corresponding to the non-repetitive C-terminal end of this 48-residue GAR/RGG region (NCL-P7; SEQ ID NO: 51) (FIG. 8A). Coated TLR2 was incubated with these four peptides at increasing concentrations and NCL-P2 was used as a positive control. NCL-P6 largely replicated NCL-P2 in TLR2 binding albeit saturation was only achieved at much higher concentrations (80 μg/ml) (FIG. 8F). For NCL-P2, saturated binding was reached at 3.2 μg/ml (FIG. 8F). NCL-4 represents the N-terminal half of NCL-P6 peptide and showed no TLR2 binding at the highest concentration tested (200 μg/ml). NCL-P5 represents the C-terminal half of NCL-P6 which exhibited a low level of TLR2 binding at 200 μg/ml (FIG. 8A). This suggests that the 20-residue NCL-P6 peptide is at the core of the NCL alarmin activity but it is not optimal. These 7 peptides were also compared in inducing cytokines from monocytes. Besides NCL-P1 and NCL-P2, only NCL-P6 induced TNFα from monocytes (FIG. 8G). The same conclusion was reached when IL-1β production was determined (FIG. 10).

### Example 6

### Alarmin Activity of Fibrillarin (FBRL) and GAR1

GAR/RGG is a common motif found at heterogenous sequence and length in nuclear proteins, including other nucleolar proteins such as the autoantigen box C/D small nucleolar RNP subunit fibrillarin (FBRL) and the box H/ACA snoRNP subunit 1 (GAR1) [Welting, T. J J., Raijmakers, R. & Pruijn, G. J., Autoimmunity Reviews 2: 313-321 (2003); Thandapani, P., et al., Mol Cell 50: 613-623 (2013)]. Based on our data on NCL, we investigated whether the GAR/RGG-motif in some other GAR/RGG-containing autoantigens had alarmin activity and could contribute to their intrinsic autoimmunogenicity. Such information may help define the molecular mechanisms underlying ANA induction in SLE and other autoimmune diseases.

FBRL is an autoantigen which contains a long GAR/RGG region close to the N-terminus (RGGGFGGRGGFGDRGGRGGRGGFGGGRGRGGGFRGRGRGG; FBRL-GAR/RGG SEQ ID NO: 5) followed by a shorter GAR/RG region. A recombinant FBRL was generated to determine whether it contains alarmin activity (FIG. 11A; SEQ ID NO: 2). The recombinant FBRL strongly induced TNFα production from PBMC (FIG. 11B). We then deleted a long GAR/RGG region from FBRL to generate the FBRL(Δ8-64)-HA mutant and this diminished FBRL induction of TNFα, suggesting that this GAR/RGG is also an alarmin motif. We similarly synthesized peptides that cover this GAR/RGG region, i.e. FBRL-P1 (SEQ ID NO: 10), FBRL-P2 (SEQ ID NO: 11) and FBRL-P3 (SEQ ID NO: 52) (FIG. 11C). FBRL-P1 and FBRL-P2 but not FBRL-P3 activated PBMC (FIG. 11C). This was not surprising because both FBRL-1 and FBRL-2 contained regular RGG-repeating sequences but FBRL-P3 is largely a poly-G sequence (FIG. 11A). Based on these data, we investigated whether GAR1 (SEQ ID NO: 3), which contains a long GAR/RGG sequence close to its C-terminus (RGGGRGGRGGGRGGGGRGGGRGGGFRGGRGGGGGGFRGGRGGG, GAR1-GAR/RGG, SEQ ID NO: 6), has alarmin activity (FIG. 11D). We generated recombinant GAR1-HA and it indeed activated PBMC (FIG. 11E). We therefore predict that more GAR/RGG-containing nuclear proteins, as elegantly summarized by Thandapani et al. (2013), exhibit alarmin activities.

Our data indicate NCL is a prototype for nuclear proteins that contain both autoimmunogenic epitopes and adjuvant signals. We have shown this to be applicable to FBRL which is a known autoantigen and contains GAR/RGG sequences. Whether GAR1 is also an autoantigen has not been determined. Their capacity to induce cytokines from PBMCs has been briefly demonstrated (FIG. 11). Numerous nuclear proteins contain the GAR/RGG or similar motifs and, if this alarmin activity is common to some of these motifs, it is not surprising that many nuclear proteins are autoantigens. Whether all or some of these GAR/RGG-containing nuclear proteins also carry epitopes for autoreactive B cells is unknown.

### Example 7

NCL-P1 and NCL-P2 Deliver Fusion Antigens into the Cytoplasm of Antigen-Presenting Cells to Induce Cytotoxin T Lymphocyte (CTL) Immunity

In conventional viral vaccine development, live-attenuated vaccines are advantageous as they retain the ability of delivering viral antigens into antigen-presenting cells which is required for effective CTL activation. To facilitate antigen entry of the cytoplasm, some researchers attempted to fuse recombinant vaccine antigens with synthetic cell-penetrating peptides (CPPs). When we study how the NCL-P2 adjuvant peptide might bind to PBMCs, we discovered an unexpected property of NCL-P2 that it penetrates the cell membrane. This makes it a rare peptide adjuvant with the dual potentials to enable fused vaccine antigens to activate TLR2 on APCs and also to cross the membrane of APCs for MHC I presentation to CD8 T cells to induce CTL immunity. NCL-P1 is also a CPP.

### The GAR/RGG Peptide NCL-P2 has Potent CPP Activity

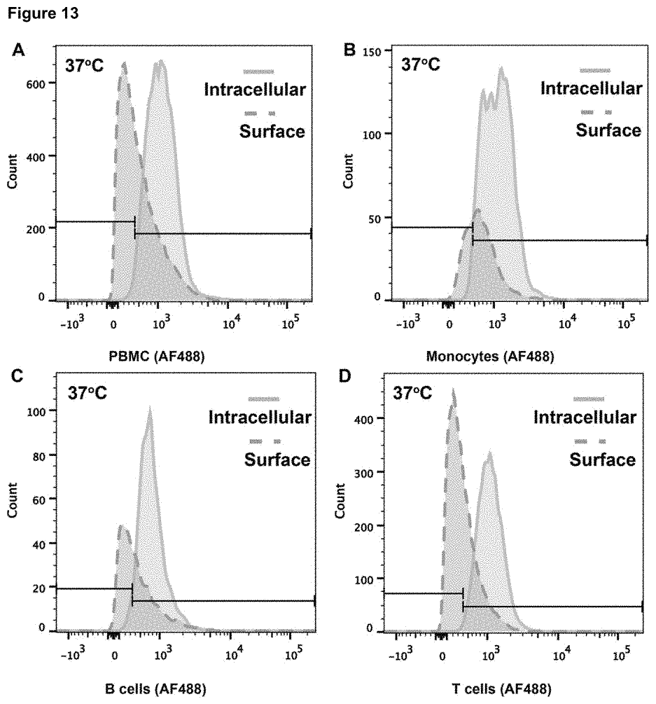

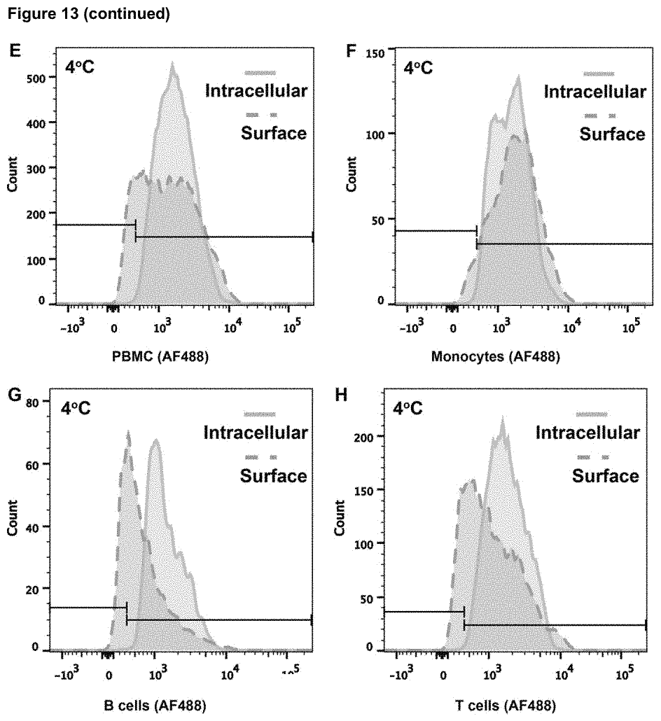

Initially, to determine how the NCL-P2 peptide may bind differently to different cell lineages in PBMC, which contains principally monocytes, B cells, T cells and natural killer cells, these cells were isolated from healthy human blood donors. The biotin-tagged NCL-P2 peptide was incubated with PBMC at 37° C. for 1 hr, then the PBMC were incubated with fluorescent lineage-specific antibodies that bind to monocytes (CD14), B cells (CD19), or T cells (CD3), respectively (FIG. 13). Dead cells were identified by incubation with the BioLegend's Zombie Cell Viability reagent (APC-Cy7). Bound NCL-P2 was detected with streptaviding-AF488 and cells were washed and analyzed by flow cytometry. Apparently, the three cell types exhibited varying levels of surface binding by NCL-P2 at 37° C., with NCL-P2 binding to the surface of the majority of monocytes and smaller fractions of B and T cells (FIG. 13). However, when cells were permeabilized, a much higher level of the peptide was detected in all PBMC suggesting prominent intracellular pools of the peptide. One explanation is the rapid endocytosis of the bound NCL-P2 peptide at 37° C. by all three cell types. It is widely accepted that monocytes are much more endocytic than B and especially T cells but similar levels of intracellular NCL-P2 peptide were detected in all three cell types, raising the question of receptor-independent, non-specific entry of the peptides into these cells.

The experiment was also performed at 4° C. which was not expected to affect surface binding but was expected to prevent endocytosis (FIG. 13). With monocytes, surface-bound NCL-P2 peptide increased significantly at 4° C. which is consistent with surface accumulation of the peptide when endocytosis was hampered. To lesser extent, NCL-P2 also accumulated on the surface T cells but no accumulation was found on B cells (FIG. 13). However, all three cell types, i.e. monocytes, B cells and T cells, continued to exhibit similarly high intracellular pools of the peptide that cannot be explained by endocytosis (FIG. 13). This is consistent with the behavior of cell-penetrating peptides (CPPs) which were initially documented with peptides in the Antennapedia transcription factor penetratin (RQIKIWFQNRRMKWKK, SEQ ID NO: 15) and the HIV protein TAT (YGRKKRRQRRR, SEQ ID NO: 16), respectively [Derossi, D. et al., J Biol Chem 269: 10444-10450 (1994); Vives, E., et al., J Biol Chem 272: 16010-16017 (1997)]. CPP penetration of the cell membrane is receptor-independent and can occur at 4° C. [Derossi, D. et al., J Biol Chem 269: 10444-10450 (1994); Derossi, D. et al., J Biol Chem 271: 18188-18193 (1996)].

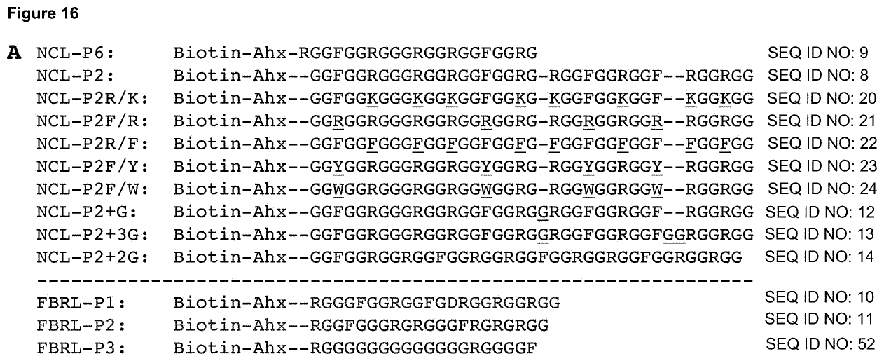

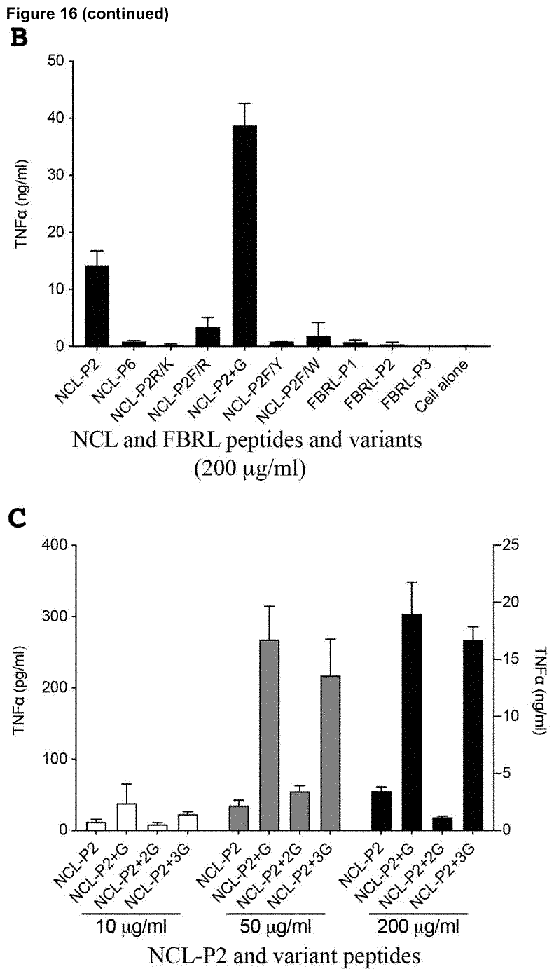

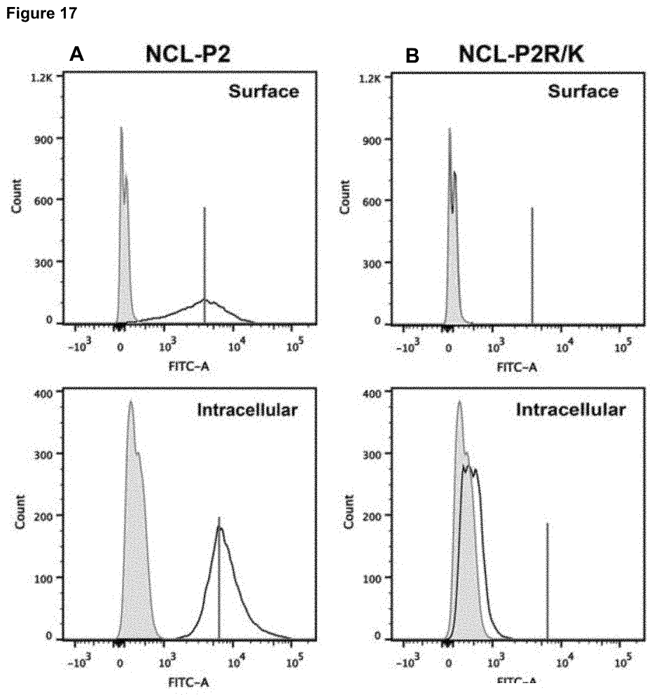

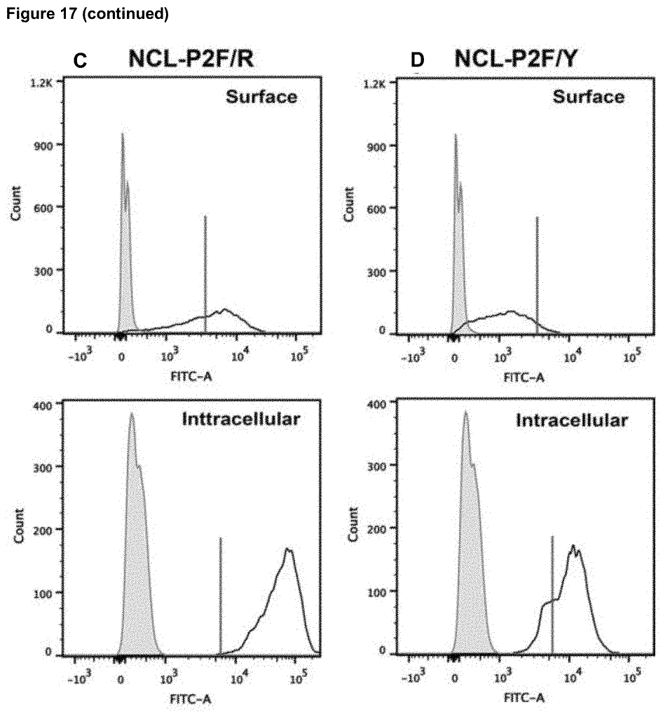

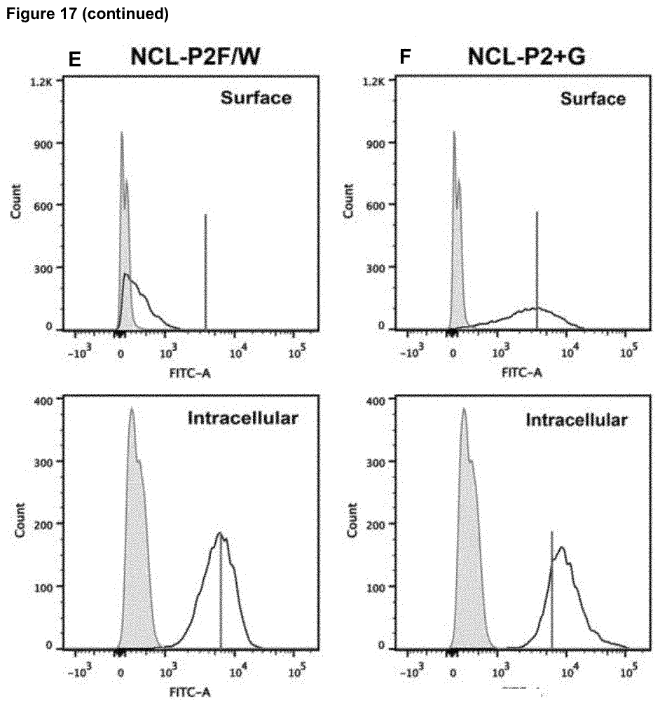

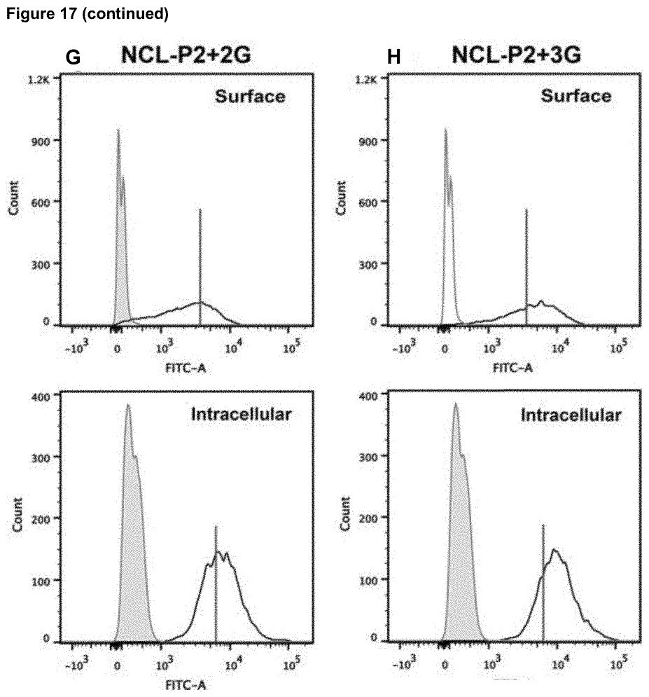

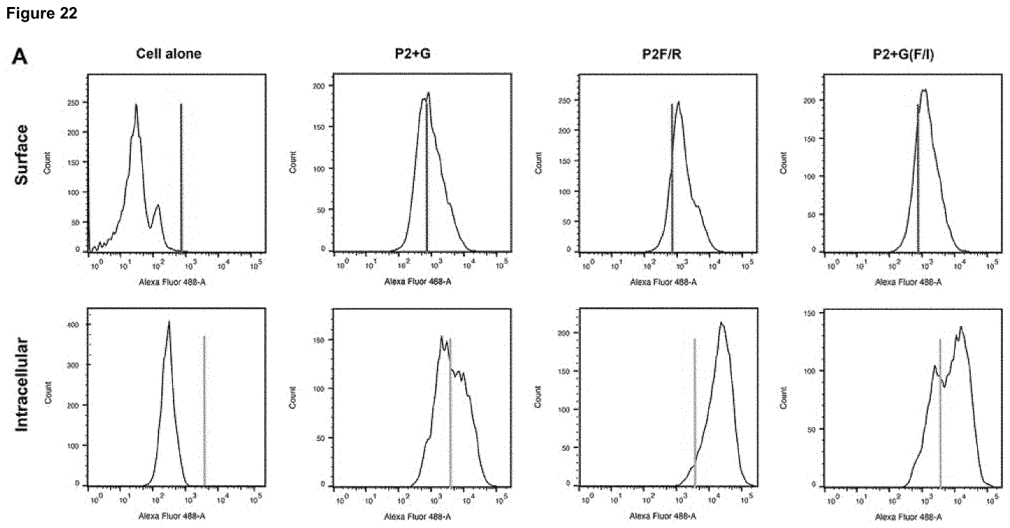

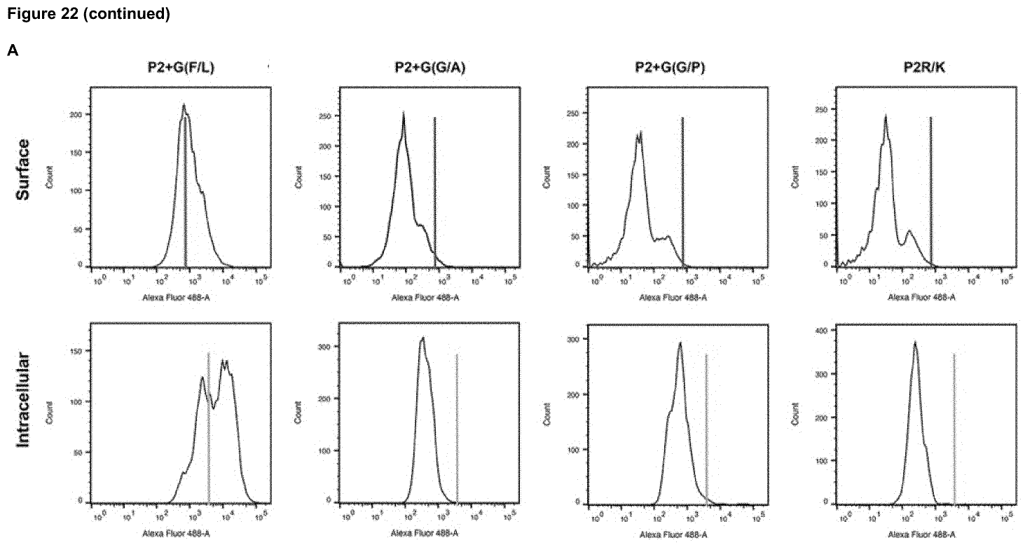

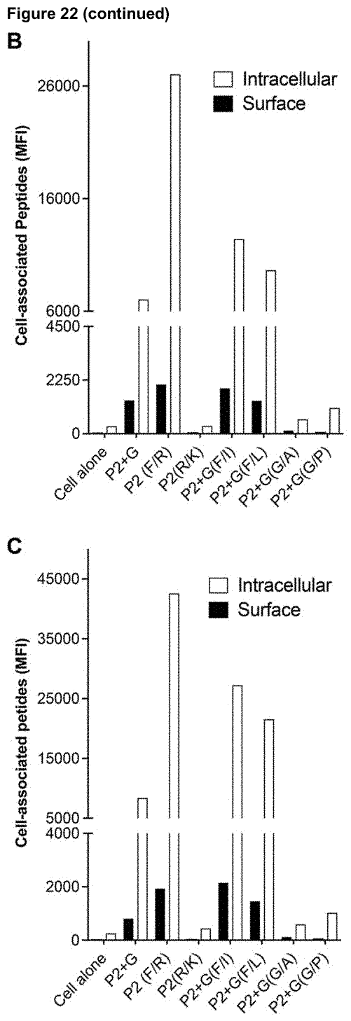

Some known cationic CPPs are characterized by the abundance of arginine (R) and lysine (K) residues [Brock, R., Bioconjug Chem 25: 863-868 (2014); Takeuchi, T. and Futaki, S., Chem Pharm Bull (Tokyo) 64: 1431-1437 (2016)]. The NCL-P2 peptide indeed contains abundant arginine (R) residues. To evaluate whether, after these arginine residues are changed to lysine residues, the peptide still retains the CPP activity, we mutated all 8 arginine into lysine residues in NCL-P2 to create a NCL-P2(R/K) mutant (FIG. 16; SEQ ID NO: 20)). Surprisingly, this mutant peptide no longer penetrates PBMC like NCL-P2 at 37° C. or 4° C. (FIG. 17B, FIG. 22), showing the critical dependence of NCL-P2 on its arginine residues for cell surface binding and cell penetration.

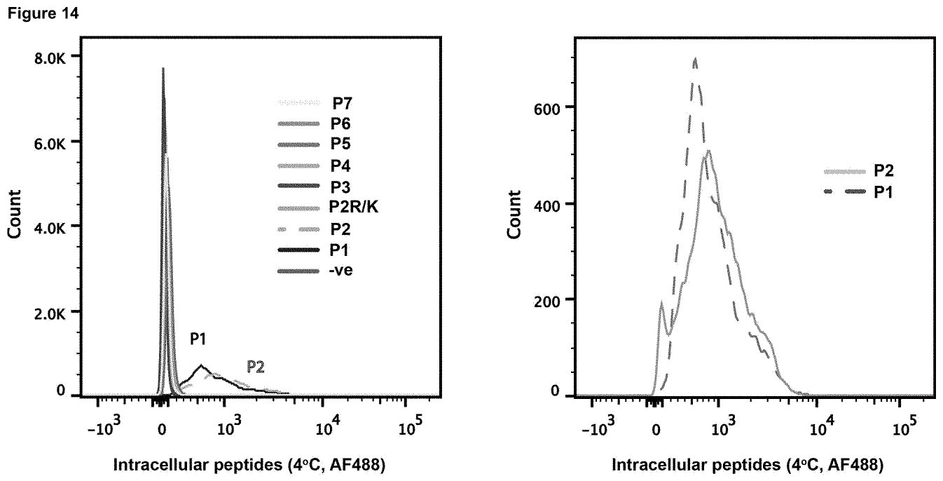

We then asked whether the NCL-P1 peptide also penetrates the cell membrane. The NCL-P1 peptide was similarly incubated with PBMCs and the cells were permeabilized to detect the intracellular pool of peptide by incubating with streptavidin-AF488 after fixation and permeabilization (FIG. 14). The NCL-P1 and NCL-P2 peptides exhibited similar cell-penetrating capacity at 4° C. (FIG. 14, right panel), so NCL-P1 peptide is also a CPP. We then similarly examined peptide P3, P4, P5, P6 and P7. P3 is a short 12-AA peptide unrelated with the GAR/RGG motif and it showed no intracellular pool (FIG. 14, left panel). The NCL-P4, NCL-P5, NCL-P6 and NCL-P7 peptides are shorter GAR/RGG peptides and all lacked cell penetration. The longest among these 4 shorter peptides is NCL-P6 which corresponds to the overlapping sequence between the NCL-P1 and NCL-P2 peptides. It retained minor adjuvant activity (FIG. 8G, FIG. 10), but showed no significant CPP activity.

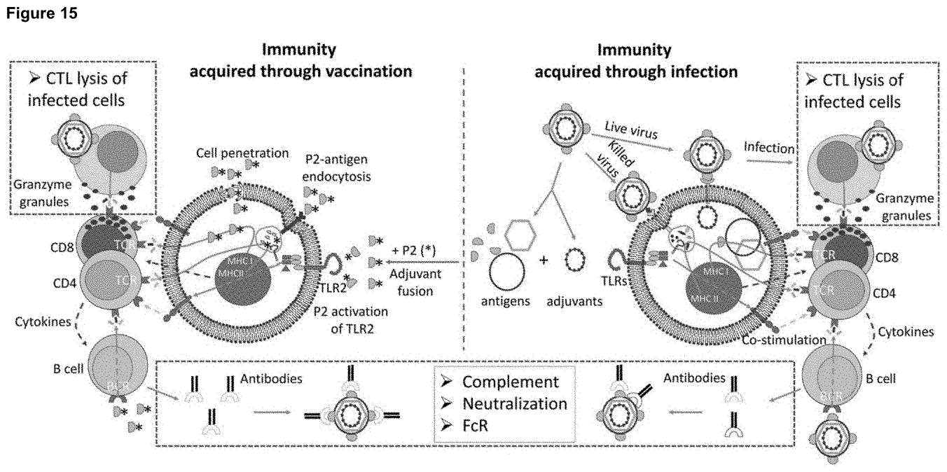

The CPP property of the NCL-P2 and NCL-P1 peptides offers another rare adjuvant activity besides their TLR2 binding and activation of APCs, which has not been found in any other TLR ligand. The simple fusion of these adjuvant peptides, especially NCL-P2, with recombinant vaccine antigens can potentially convert isolated vaccine antigens into ‘molecular viruses’ that: 1) carry B and T cell epitopes to induce protective antibodies and T cells, 2) contain a TLR2 ligand that activate APCs and CD4 T cells that help in B and T activation, and 3) ‘infect’ APCs so vaccine antigens can be delivered to the cytoplasm for MHC I presentation to CD8 T cells and the generation of CTLs (FIG. 15). Therefore, the dual adjuvant property of the NCL-P2 peptide overcomes a major technical barrier to developing recombinant viral or cancer proteins into powerful vaccines that induce humoral and cellular immunity like live virus-based or live-attenuated viral vaccines (FIG. 15), a property that is lost in inactivated viral vaccines.

### Example 8

### NCL-P2 can Gain or Lose Adjuvant and CPP Activities Through Changes in its Sequence

Besides NCL-P2, other GAR/RGG sequences also exhibited adjuvant activity (FIG. 11). The R/K mutant of NCL-P2 lost its adjuvant activity (FIG. 16). We then synthesized a series of mutant NCL-P2 peptides to determine whether some amino acids could be substituted to affect its adjuvant activity and found that some changes increased its adjuvant activity. The eight NCL-P2 mutants are listed in FIG. 16A but the mutant in which all arginine were changed to phenylalanine residues (NCL-P2R/F, SEQ ID NO: 22) was not successfully synthesized. The 7 successfully synthesized NCL-P2 mutants were tested for their gain or loss of adjuvant activity by stimulation of PBMC. Apart from the high percentage of glycine residues (G), the regularly spaced R and F residues were the only other residues in the NCL-P2 sequence (FIG. 16A). As shown in FIG. 16B, replacement of the R residues with the most closely-related K residues in NCL-P2 (SEQ ID NO: 20) completely abolished its adjuvant activity, and changing the F residues to the closely related Y (SEQ ID NO: 23) or W (SEQ ID NO: 24) residues also markedly diminished its adjuvant activity. Replacing all the F residues with R residues (SEQ ID NO: 21) substantially reduced its adjuvant activity. These data suggest an essential requirement for specific R and F composition and positions for NCL-P2 to display adjuvant activities.

We then changed an irregular ‘RGRGG’ sequence in NCL-P2 into the regular ‘RGGRGG’ sequence found in the rest of the peptide by adding one ‘G’ residue. This NCL-P2+G (SEQ ID NO: 12) mutant peptide exhibited a 3-fold increase in adjuvant activity as compared with the wild type NCL-P2 peptide (FIG. 16B). We then attempted further to make the RGGFRGG′ sequence in the NCL-P2+G mutant a regular ‘RGGFGGRGG’ sequence. This NCL-P2+3G mutant peptide (SEQ ID NO: 13) showed no further improvement in adjuvant activity and instead it slightly reduced the high adjuvant activity of the NCL-P2+G mutant peptide (FIG. 16C). At the same time, we also speculated that the increased adjuvant activity in NCL-P2+G was due to the more regular FGGRGGRGG sequence created by introducing the additional ‘G’ residue and we therefore re-organized the sequence of NCL-P2 into basically four repeats of ‘FGGRGGRGG’. Compared with the wild type NCL-P2 peptide, this NCL-P2+2G mutant peptide (SEQ ID NO: 14) showed diminished rather than increased adjuvant activity, suggesting intrinsic sequence determinants in NCL-P2 that confers adjuvant activity which can be enhanced as in NCL-P2+G.

The 7 NCL-P2 also exhibited gain or loss in CPP activity (FIG. 17). The NCL-P2R/K mutant (SEQ ID NO: 20) lost CPP as well as alarmin activity. The NCL-P2F/R mutant (SEQ ID NO: 21) gained substantial CPP activity while losing significant alarmin activity (FIG. 17C). The NCL-P2F/Y mutant (SEQ ID NO: 23) gained significant CPP activity but its alarmin activity was diminished (FIG. 17D). The NCL-P2F/W mutant (SEQ ID NO: 24) retained the CPP activity but it lost the alarmin activity (FIG. 17E). The NCL-P2+G (SEQ ID NO: 12) and NCL-P2+3G (SEQ ID NO: 13) both showed slightly higher CPP activity but both gained significantly higher alarmin activity (FIGS. 17F and H, respectively). The NCL-P2+2G (SEQ ID NO: 14) mutant was similar to NCL-P2 in CPP activity but it has slightly reduced alarmin activity (FIG. 17G). The NCL-P2R/F mutant (SEQ ID NO: 22) has not been able to be synthesized successfully so it was not examined (FIG. 16A).

### Example 9

### 9.1 Peptide P2+G can Penetrate Dendritic Cells (DC)

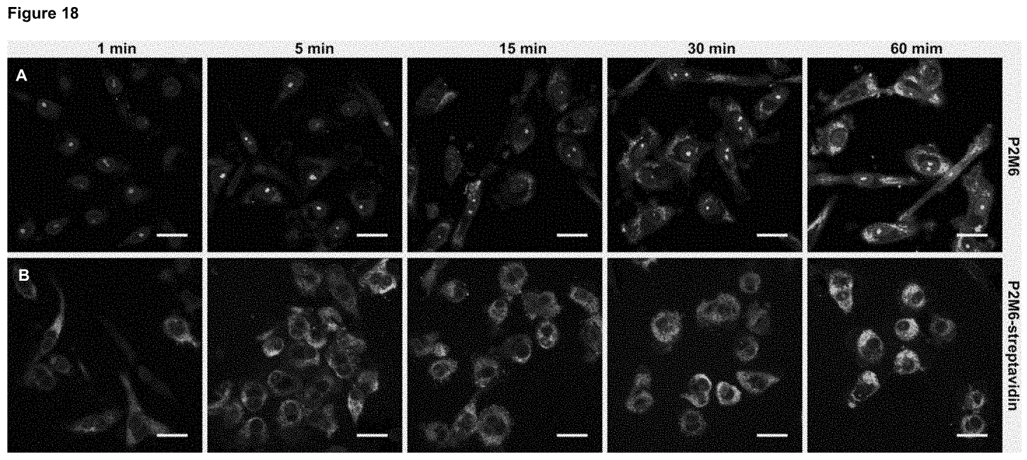

DC are essential to the host translating vaccines into effective immunity [Steinman and Hemmi, 2006]. If P2+G peptide can penetrate DC, it potentially delivers vaccine antigens into DC cytoplasm for MHC class I presentation to CD8 T cells [Blum, J. S., Wearsch, P. A., Cresswell, P., Annu Rev Immunol 31:443-473, (2013)]. DC were cultured from monocytes that were isolated from healthy blood donors [as described in Example 1 and Cao, W., et al., Blood 107: 2777-2785 (2006), incorporated herein in its entirety]. On coverslips, DC were incubated for 1 to 60 min (1, 5, 15, 30 and 60 min) at 4° C. with P2+G (200 μg/ml). After fixation and permeabilization, the cells were incubated with streptavidin-Alexa Fluor 488 (AF488) (Thermo Fisher Scientific, Waltham, Mass.) and, after washing, mounted with DAPI-containing media and examined by confocal microscopy. As shown in FIG. 18A, P2+G penetrated DC within 1 min of incubation and the intracellular pool of P2+G increased progressively from 5 to 60 min. The peptide penetrated into the nucleoli faster than it penetrated into the cytoplasm.

9.2 P2+G Carries Streptavidin Across the Membrane into the DC Cytoplasm

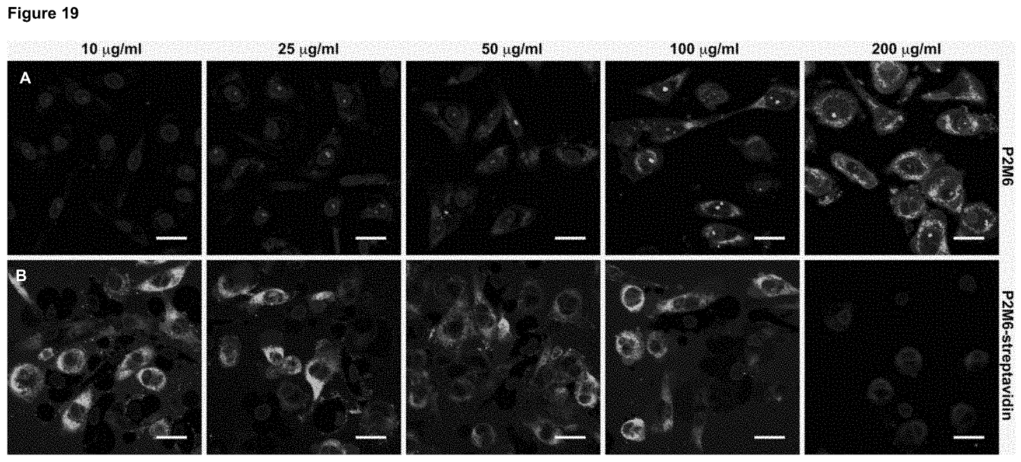

P2+G was also first incubated with streptavidin-AF488 for 30 min on ice to form the streptavidin-P2+G conjugates. These pre-formed conjugates were then incubated with DC on coverslips without prior fixation or permeabilization. Cells were washed, fixed and directly mounted without permeabilization for confocal microscopy analysis. As seen with P2+G, the P2+G-streptavidin conjugates also rapidly penetrated DC (FIG. 19B). Streptavidin is a tetrameric protein of approx. 56 kDa. However, unlike P2+G, the P2+G-streptavidin conjugates no longer concentrate in the nucleoli. Instead, they localized predominantly in the cytoplasm.

It was also observed that the P2+G-streptavidin conjugates penetrated DC much faster than the P2+G peptide, reaching saturation within 5 min (FIG. 18B). The P2+G peptide only reached saturation in DC cytoplasm after 30 min (FIG. 18A). We consider that each streptavidin binds multiple P2+G peptide which could have enhanced P2+G binding to and translocation across the cell membrane. Nonetheless, P2+G can carry cargo proteins across the cell membrane into the cytoplasm.

### 9.3 P2+G Only Effectively Penetrates DC at Higher Peptide Concentrations

The P2+G peptide is, in general, cationic and representatives of this category of peptides include oligoarginine peptides of varying lengths [Mitchell, D. J., et al., J Pept Res 56: 318-325 (2000)]. Mechanistically, it has been suggested that oligoarginine peptides first concentrate on the cell membrane like CaCl₂) and then translocate across the membrane by induced membrane re-organization [Mitchell, D. J., et al., J Pept Res 56: 318-325, (2000); Allolio, C. et al., Proc Natl Acad Sci USA 115: 11923-11928 (2018)]. To examine whether P2+G concentration impacts on its cell penetration, DC were then incubated with different concentrations of P2+G (10, 25, 50, 100, or 200 μg/ml). When P2+G was incubated with DC, penetration was not detectable at 10 μg/ml (FIG. 19A). A low level of DC penetration was observed at 25 μg/ml of P2+G (FIG. 19). The intracellular pool of P2+G increased following increasing concentrations of the peptide from 25 to 200 μg/ml (FIG. 19A).

9.4 after Binding to Streptavidin, P2+G Penetrated DC at Much Lower Peptide Concentrations

When different concentrations of P2+G (10, 25, 50, 100 and 200 μg/ml) were bound to streptavidin-AF488 (50 μg/ml) for 30 min on ice before incubation with DC, prominent DC penetration at 10 μg/ml was observed (FIG. 19B). At this concentration, free P2+G showed no detectable DC penetration (FIG. 19A). In fact, P2+G-streptavidin penetration of DC apparently reached saturation at 10 μg/ml of P2+G (FIG. 19B). Increasing P2+G from 10 to 100 μg/ml didn't further increase its intracellular pool in DC (FIG. 19B). It is likely that saturated DC penetration could have been achieved at even lower P2+G concentrations.

It was surprising that, when streptavidin (50 μg/ml) was incubated with P2+G at 200 μg/ml, the conjugates no longer penetrate DC effectively for which we do not have an explanation at this time (FIG. 19B, right panel).

### Example 10

The Generation of Two More P2+G Mutant Peptides with Strong Alarmin and CPP Activities

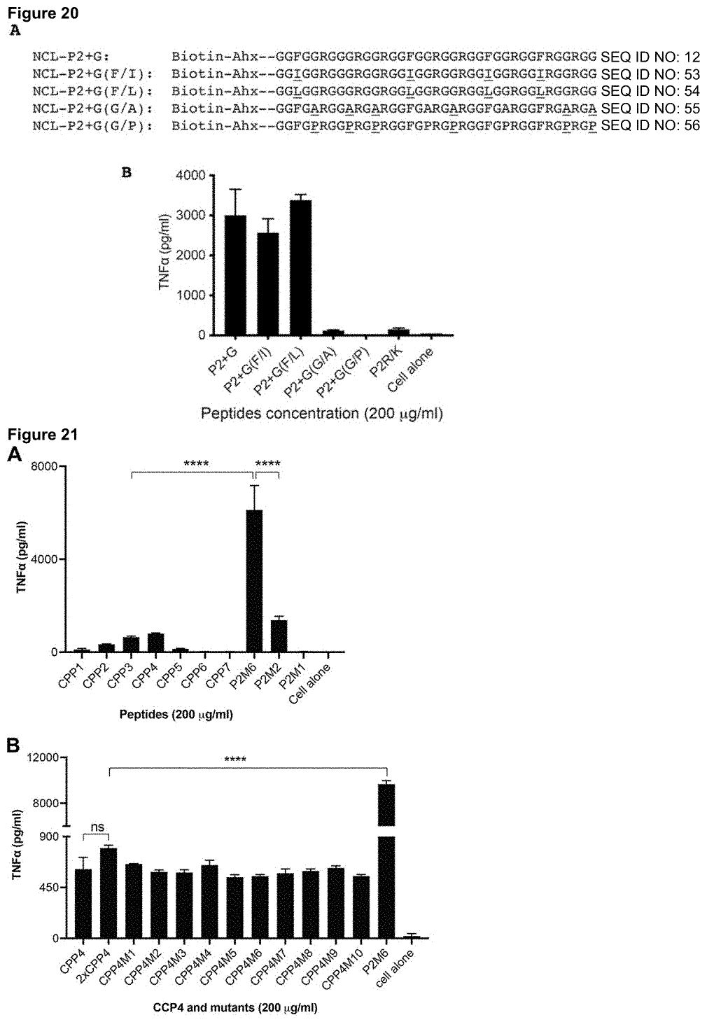

As shown in Example 8, adding one glycine to NCL-P2, a P2+G peptide with 3-fold increase in alarmin activity was generated. Further modifications of P2+G were made to determine whether further increases in alarmin or CPP activity could be achieved. Two mutant peptides were synthesized based on P2+G by changing its 4 phenylalanine residues into isoleucine (P2+G(F/I); SEQ ID NO: 53) or leucine (P2+G(F/L); SEQ ID NO: 54) residues and two more P2+G mutant peptides were synthesized by changing 6 of its 25 glycine residues into alanine (P2+G(G/A); SEQ ID NO: 55) or proline (P2+G(G/P); SEQ ID NO: 56) residues (FIG. 20A). These four new peptides were used to stimulate PBMC for 24 hr and TNFα production was measured by ELISA. P2+G and P2R/K were used as positive and negative controls. The two phenylalanine mutant peptides both exhibit comparable alarmin activity as P2+G (FIG. 20B). In fact, P2+G(F/L) appeared to exhibit stronger alarmin activity than P2+G (FIG. 20B). In contrast, the two glycine-based mutants completely lost their alarmin activities (FIG. 20B).

### Example 11

### Alarmin Activity of Other Known CPPs

Besides the NCL-derived CPPs disclosed herein, many other CPPs have been identified in previous studies. We asked whether these other known CPPs might also exhibit alarmin activity. Seven of the most studied CPPs were synthesized, i.e. CPP1-CPP7 (Table 1). These CPPs were compared with P2+G, P2F/R and P2R/K in PBMC stimulation for 24 hr followed by measuring TNFα induction using ELISA (FIG. 21A). No significant TNFα was induced by CPP1 (Tat)(SEQ ID NO: 28), CPPS (pVEC)(SEQ ID NO: 32), CPP6 (TP10)(SEQ ID NO: 33), or CPP7 (M918)(SEQ ID NO: 34), but low levels of TNFα was induced by CPP2 (penetratin)(SEQ ID NO: 29), CPP3 (oligoarginine)(SEQ ID NO: 30) and CPP4 (FHV)(SEQ ID NO: 31), especially CPP3 and CPP4 (FIG. 21A). CPP3 is an artificial peptide consisting of 16 arginine residues and CPP4 is a 15-residue peptide among which 11 residues are arginine.

Both CPP3 and CPP4 are half of the length of the 36 residue NCL-P2 and the 37 residue P2+G. In our published studies, shorter peptides inside NCL-P2 were synthesized but all the shorter peptides showed diminished alarmin activity [Wu, S., et al., Cell Death Dis 12: 477 (2021)]. These shorter peptides, including the 21 residue NCL-P6, also showed diminished CPP activities (FIG. 14).

Whether increasing the length of CPP4, by synthesizing two tandem CPP4 repeats (2×CPP4), would alter its alarmin activity was investigated. Little increase in alarmin activity was observed in 2×CPP4 (SEQ ID NO: 35) as compared with CPP4 (FIG. 21B). Since mutations in NCL-P2 generated mutant peptides with increased alarmin activities, we synthesized 10 CPP4 mutants by deletions and point mutations (CPP4M1-CPP4M10; SEQ ID NOs: 36-45, respectively) (Table 1). None of these CPP4 mutants showed significant increase or decrease in alarmin activity relative to the low CPP4 alarmin activity (FIG. 21B). Therefore, NCL-P2 remains unique for its prominent dual alarmin and CPP activities and the plasticity of both activities which could be separately increased by introducing mutations.

TABLE 1
Selected CPPs and CPP4 mutant peptides

| | | Other | SEQ ID |
|---|---|---|---|
| Peptides | Sequences | names | NO: |
| CPP1 | GRKKRRQRRRPPQ | Tat | 28 |
| CPP2 | RQIKIWFQNRRMKWKK | Penetratin | 29 |
| CPP3 | RRRRRRRRRRRRRRRR | Oligoarginine | 30 |
| CPP4 | RRRRNRTRRNRRRVR | FHV | 31 |
| CPP5 | LLIILRRRIRKQAHAHSK | pVEC | 32 |
| CPP6 | GWTLNSAGYLLGKINLKALAALAKKIL | TP10 | 33 |
| CPP7 | MVTVLFRRLRIRRACGPPRVRV | M918 | 34 |
| 2xCPP4 | RRRRNRTRRNRRRVRRRRRNRTRRNRRRVR | n.a. | 35 |
| CPP4M1 | --RRNRTRRNRRRVR | n.a. | 36 |
| CPP4M2 | RRRR--TRRNRRRVR | n.a. | 37 |
| CPP4M3 | RRRRNR--RNRRRVR | n.a. | 38 |
| CPP4M4 | RRRRNRTRR--RRVR | n.a. | 39 |
| CPP4M5 | RRRRNRTRRNRRR-- | n.a. | 40 |
| CPP4M6 | RRRRNRLRRNRRRLR | n.a. | 41 |
| CPP4M7 | RRRRLRLRRLRRRLR | n.a. | 42 |
| CPP4M8 | RRRRNRNRRNRRRNR | n.a. | 43 |
| CPP4M9 | RRRRRRTRRRRRRVR | n.a. | 44 |
| CPP4M10 | RRRRNRRRRNRRRRR | n.a. | 45 |

2xCPP4 consists of two tandem CPP4 sequences with one being underlined. '-', residue deletions. n.a., not available.

### Example 12

### The Two Phenylalanine Mutants of P2+G Retained the CPP Activity but the Two Glycine Mutants of P2+G Completely Lost CPP Activity

After the 4 new P2+G mutant peptides were examined for their alarmin activities (FIG. 20), they were also tested for their CPP activities.

PBMC were incubated for 1 hr at 4° C. separately with each of the four P2+G mutant peptides. The mutations involved either the phenylalanine or glycine residues in P2+G. The 4 phenylalanine residues in P2+G were changed either to isoleucine (P2+G(F/I)) or leucine (P2+G(F/L)). Six of the 25 glycine residues in P2+G were changed to either alanine (P2+G(G/A)) or proline (P2+G(G/P)) residues. These peptides (200 mg/ml) were incubated with PBMC for 1 hr at 4° C. Surface-bound and intracellular peptides were detected with streptavidin-AF488. As controls, PBMC were incubated with P2+G, P2F/R, or P2R/K. Cells were analysed by flow cytometry (FIG. 22A). The vertical bars were used to indicate the surface and intracellular fluorescence intensity obtained with P2+G which were used as references for those of other peptides. Based on the flow cytometry results in FIG. 22A, mean fluorescence indices (MFI) were calculated and compared (FIG. 22B). PBMC were also incubated for 1 hr at 37° C. with the peptides and similarly examined by flow cytometry and MFI calculated (FIG. 22C). Only MFI data are shown for these experiments.

The two glycine mutants of P2+G, i.e. P2+G(G/A) and P2+G(G/P), which lost the alarmin activity (FIG. 20B), also completely lost the CPP activity (FIG. 22A-C). It shows that the number and arrangement of the glycine residues in NCL-P2 and its P2+G mutant are essential to their alarmin and CPP activities. In contrast, the two phenylalanine mutants of P2+G, i.e. P2+G(F/I) and P2+G(F/L), which retained the alarmin activity (FIG. 20B), acquired higher CPP activities (FIGS. 22 B and C). Therefore, P2+G(F/I) and P2+G(F/L), together with P2+G and P2+3G, constitute a group of peptide adjuvants for vaccine development. PBMC penetration by these 4 P2+G mutant peptides were examined at 4° C. (FIGS. 22A and 22B) and 37° C. (FIGS. 22A and 22C) and similar results were obtained.

### Example 13

### The P2+G Peptide can Activate DC Maturation

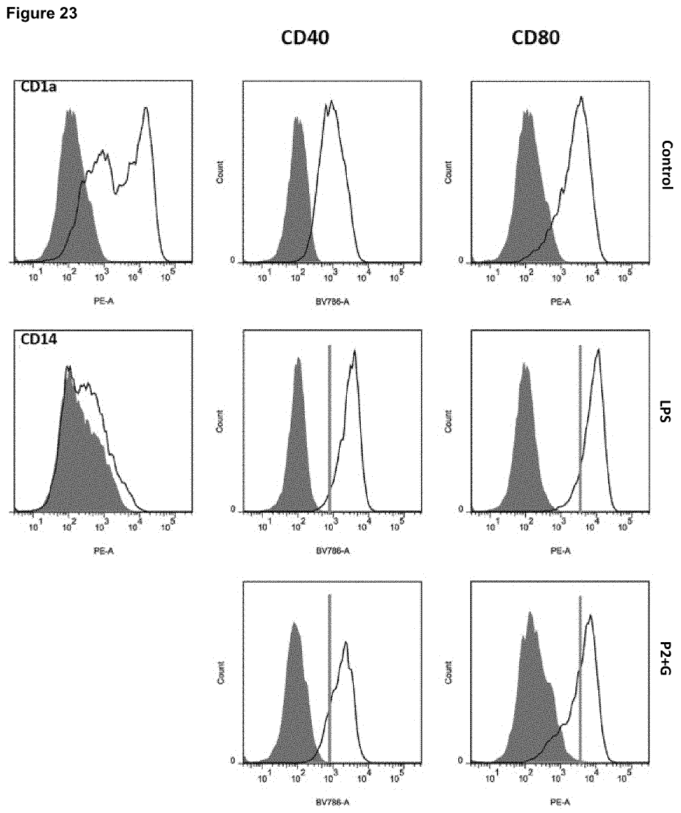

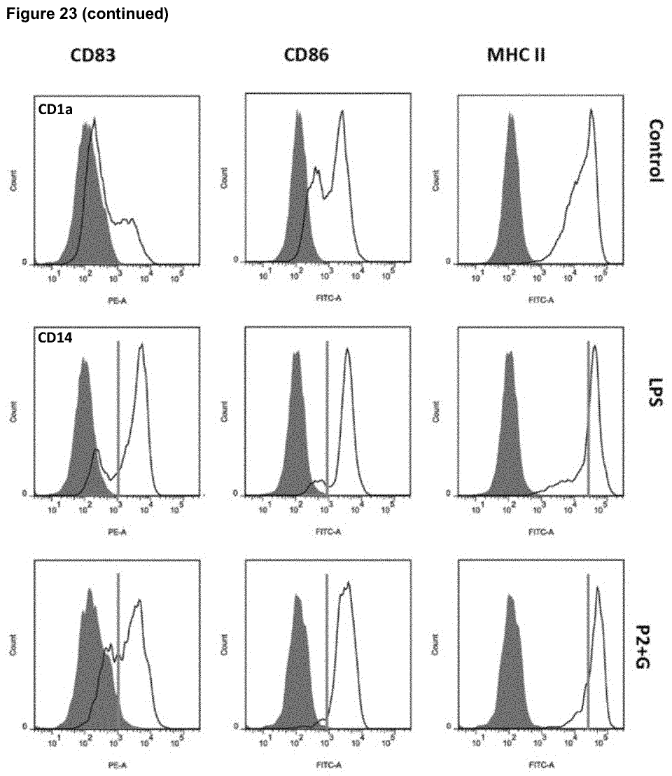

NCL-P2 was previously shown to activate DC as judged by TNFα induction [Wu, S., et al., Cell Death Dis 12: 477 (2021)]. Whether P2+G effectively activates these cells into mature antigen-presenting cells (APC) for effective T cell activation has not been examined. To this end, DC were stimulated for 48 hr with P2+G (200 μg/ml) or, as a positive control, LPS (0.5 μg/ml). As a negative control, DC were cultured without specific stimulation by adding equivalent volumes of PBS. DC were cultured from monocytes and were typically CD14^{lo/-} CD1a^{hi} [Cao, W., et al., Blood 107: 2777-2785 (2006)]. Cells were examined for surface expression of MHC class II, CD40, CD80, CD83 and CD86. As shown in FIG. 23, P2+G effectively activated DC maturation because it up-regulated all these molecules on DC. This suggests that P2+G is a likely to be a sufficiently potent vaccine adjuvant.

### Example 14

Dendritic Cells Activate Autologous Human CD4 and CD8 T Cells when Exposed to a Fusion Polypeptide Comprising P2+G and a 30-AA Peptide Antigen IPA1E2

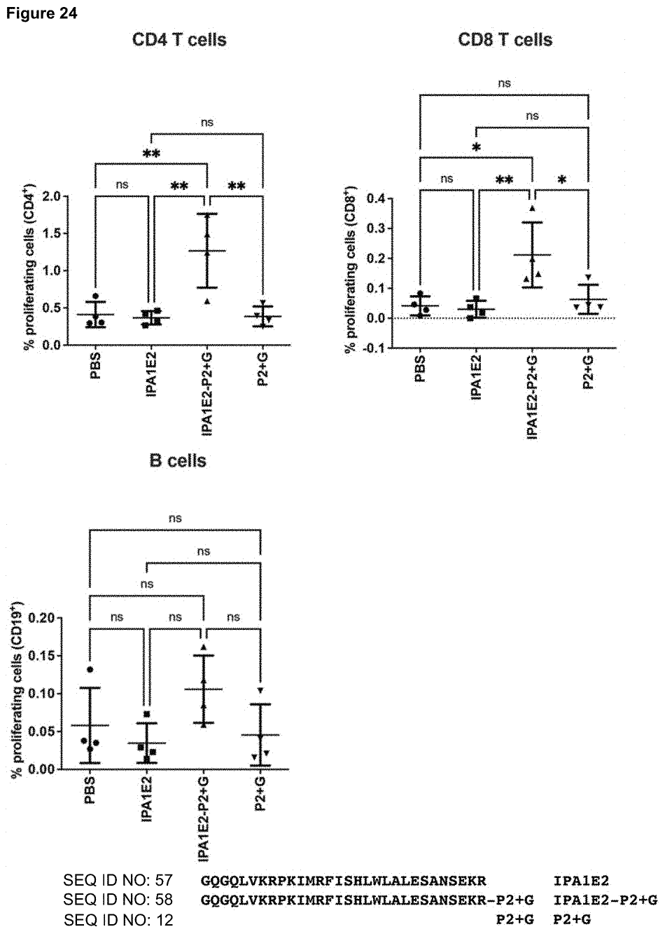

To evaluate whether the P2+G peptide confers significant adjuvant activity to the antigen to which it is fused, P2+G was synthesized in fusion with a 30-AA peptide antigen (IPA1E2; SEQ ID NO: 57) (FIG. 24) by ChemPeptide (Shanghai, China). P2+G and IPA1E2 were also synthesized as separate peptides.

PBMC were isolated from healthy blood donors from which monocytes were isolated to culture DC and the remaining cells (mostly lymphocytes) were stored frozen as a source of autologous lymphocytes. DC were then incubated for 24 hr with P2+G, IPA1E2 or the IPA1E2-P2+G fusion polypeptide (SEQ ID NO: 58) in round bottom 96-well plates (5×10⁴ cells/well) without adding additional adjuvants. The frozen lymphocytes were revived and labelled with CellTrace Violet (2.5 μM) for 10 min at 37° C. and then added to the DC wells at 2.5×10⁵ cells/well (DC:T ratio=1:5). After 2 weeks of co-culture, the proliferation of CD4+ and CD8+ T cell and CD19+ B cell was analyzed by flow cytometry. As shown in FIG. 24, DC loaded with the IPA1E2-P2+G fusion antigen caused significantly increased T cell proliferation than that induced by individual IPA1E2 or P2+G peptides. It shows that the P2+G portion might not induce significant T cell activation to itself (low antigenicity) but it enabled the IPA1E2 antigen to activate T cells.

### Example 15

### NCL-P2 and its Mutant Peptides Show Little Cytolytic Activity

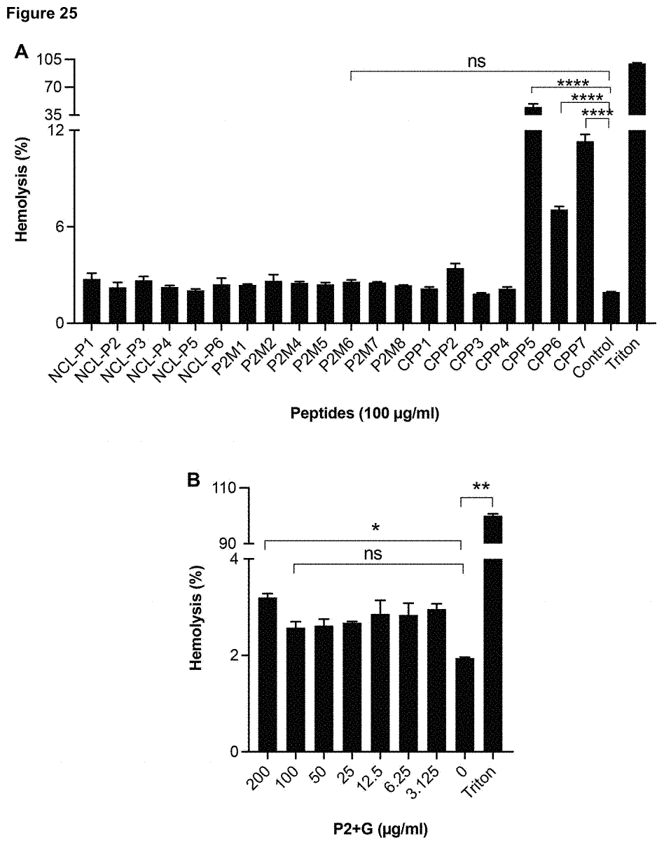

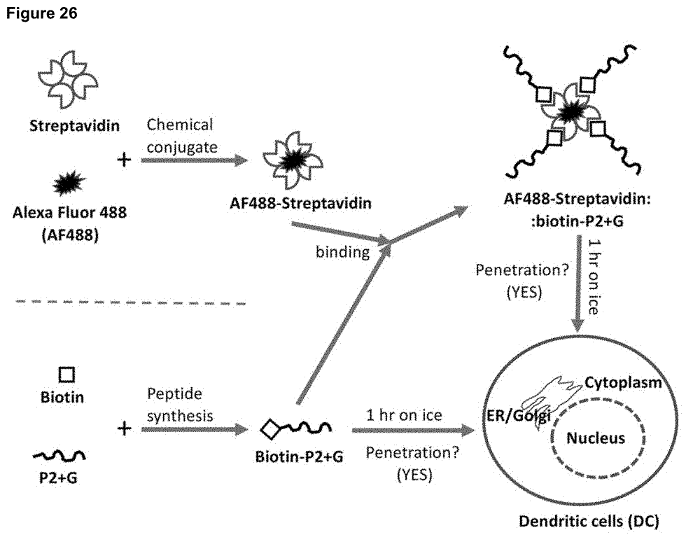

To use NCL-P2 and its mutant peptides in vaccines or drug delivery, one potential concern is whether they exhibit cytotoxicity to host cells. The CPP activities of these peptides presented apparent concerns whether they cause cytolysis. We then tested the cytolytic activity of these peptides using a haemolytic assay, which was based on the lysis of red blood cells [Evans, B. C. et al., J Vis Exp, e50166, (2013)]. It was clear that all NCL-P2 related peptides, including its 11 mutants, caused insignificant haemolysis above the PBS control (FIG. 25A). Among the 7 other known CPPs, CPP1-4 caused little haemolysis but CPPS-7 were clearly haemolytic, especially CPP5 (FIG. 25A).

With P2+G, haemolysis was also examined at different peptide concentrations (3.125-200 μg/ml). Similar low background haemolysis was observed at all concentrations suggesting that peptide-specific haemolysis is absent (FIG. 25B). It shows that, while some other known CPPs were indeed haemolytic or cytolytic, NCL-P2 and its mutant peptides can effectively penetrate cell membrane without leading to cell lysis. No significant cytotoxicity was detected in NCL-P2 and its mutant peptides when the Zombie (NIR) Fixable viability stain-APC-Cy7 assay or the CELLTITER 96® AQueous One Solution Cell Proliferation (MTS) Assay were used to detect cytotoxicity of these peptides (data not shown). This removes a major safety concern when these peptides are explored in a broad range of biomedical and clinical applications.

### SUMMARY

We set out to understand what made the nucleolus highly autoimmunogenic [Beck, J. S., Lancet 1: 1203 (1961); Welting, T. J., Raijmakers, R. & Pruijn, G. J., Autoimmunity Rev 2:313, (2003); Cai, Y., et al., J Biol Chem 290: 22570 (2015); Cai, Y. et al. J Immunol 199: 3981 (2017)], and discovered that a major nucleolar autoantigen nucleolin (NCL) contained potent alarmin activity [Wu, S., et al., Cell Death Dis 12:477, (2021)]. Within nucleolin (NCL), we localized alarmin activity to its 48 amino acid long GAR/RGG motif. A 36-amino acid peptide within this motif, i.e. NCL-P2, replicated NCL in the activation of PBMC and other immune cells. For both NCL and NCL-P2, the major receptor is TLR2 with TLR4 being also likely to be involved [Wu, S., Teo, B. H. D., Wee, S. Y. K., Chen, J. & Lu, J., Cell Death Dis 12:477, (2021)]. The strong alarmin activity of NCL-P2 made it a potential adjuvant for vaccines.

The surprising discovery of a potent CPP activity in NCL-P2 makes it a unique vaccine adjuvant which can potentially carry cargo antigens into antigen-presenting cells (APC) while simultaneously activate these cells for T cell activation. Delivery of antigens inside APCs is key to effective activation of vaccine antigen-specific CD8 T cells into CTLs. Extensive mutagenesis of NCL-P2 showed that its alarmin and CPP activities could be improved independently and substantially in specific NCL-P2 mutants, i.e. P2F/R showed reduced alarmin activity but approx. 8-fold increase in CPP activity. P2+G, P2+3G, P2+G(F/I), and P2+G(F/L) acquired 2-5 folds higher alarmin activity while also slightly increased their CPP activities.

In one example, P2+G was shown to penetrate DC and carry a cargo protein streptavidin into the DC cytoplasm (FIGS. 18 and 19). In another example, P2+G carried ovalbumin into DC cytoplasm (data not shown). Most vaccines that target intracellular pathogens or cancers are most effective when the vaccine antigens can be delivered to the cytoplasm of DC and other antigen-presenting cells (APC) for MHC I-mediated CD8 T cell activation into cytotoxic T lymphocytes (CTL). It has not been tested, but P2+3G, P2+G(F/I), P2+G(F/L), and probably other known and unexplored NCL-P1 and NCL-P2 mutants also penetrate DC and other APC and, in association with the different vaccine antigens, they carry these cargo antigens into the APC.

The strong CPP but diminished alarmin activities of P2F/R implies that it would not cause severe inflammatory responses during delivery of a drug cargo or label to a cell.

A common concern in using CPPs in vaccine development and drug delivery is whether they cause cell lysis when they penetrate the cell membranes. Three lines of studies have been performed to evaluate the cytotoxic or cytolytic activities of NCL-P2 and its mutant peptides and they all lacked detectable cytolytic and cytotoxic activity. This removes a major concern over their use in vaccines, immunotherapies, drug delivery, etc.

Overall, NCL-P2 and especially its known mutants P2+G, P2+3G, P2+G(F/I), P2+G(F/L) and P2F/R, provide a powerful series of bioactive peptides with the dual activities on one peptide, i.e. alarmin and CPP, which are highly desirable for as vaccine adjuvants or carrier for intracellular delivery of drugs or labels. The anticipated low antigenicity of these peptides based on epitope prediction (data not shown) and experimental indications (FIG. 24) lower another common safety/efficacy concern over the use of these peptides in patients.

### REFERENCES

Any listing or discussion of an apparently prior-published document in this specification should not necessarily be taken as an acknowledgement that such document is part of the state of the art or is common general knowledge.
Ahmed, S. F., Quadeer, A. A. & McKay, M. R. Preliminary Identification of Potential Vaccine Targets for the COVID-19 Coronavirus (SARS-CoV-2) Based on SARS-CoV Immunological Studies. Viruses 12, doi:10.3390/v12030254 (2020).
Allolio, C. et al. Arginine-rich cell-penetrating peptides induce membrane multilamellarity and subsequently enter via formation of a fusion pore. Proc Natl Acad Sci USA 115, 11923-11928, doi:10.1073/pnas.1811520115 (2018).
Baumann, I. et al. Impaired uptake of apoptotic cells into tingible body macrophages in germinal centers of patients with systemic lupus erythematosus. Arthritis Rheum 46, 191-201, doi: 10.1002/1529-0131(200201)46:1<191::AID-ART10027>3.0.CO; 2-K (2002).
Beck, J. S. Variations in the morphological patterns of “autoimmune” nuclear fluorescence. Lancet 1, 1203-1205 (1961).
Blum, J. S., Wearsch, P. A. & Cresswell, P. Pathways of antigen processing. Annu Rev Immunol 31, 443-473, doi:10.1146/annurev-immunol-032712-095910 (2013).
Bondanza, A. et al. Requirement of dying cells and environmental adjuvants for the induction of autoimmunity. Arthritis Rheum 50, 1549-1560, doi:10.1002/art.20187 (2004).
Brock, R. The uptake of arginine-rich cell-penetrating peptides: putting the puzzle together. Bioconjug Chem 25, 863-868, doi:10.1021/bc500017t (2014).
Cai, Y., Teo, B. H., Yeo, J. G. & Lu, J. C1q protein binds to the apoptotic nucleolus and causes C1 protease degradation of nucleolar proteins. J Biol Chem 290, 22570-22580, doi: 10.1074/jbc.M115.670661 (2015).
Cai, Y. et al. Broad Susceptibility of Nucleolar Proteins and Autoantigens to Complement Cl Protease Degradation. J Immunol 199, 3981-3990, doi:10.4049/jimmunol.1700728 (2017).
Cao, W., Tan, P., Lee, C. H., Zhang, H. & Lu, J. A transforming growth factor-beta-induced protein stimulates endocytosis and is up-regulated in immature dendritic cells. Blood 107, 2777-2785, doi:10.1182/blood-2005-05-1803 (2006).
Chen, F. et al. Neoantigen Identification Strategies Enable Personalized Immunotherapy in Refractory Solid Tumors. J Clin Invest 129, 2056-2070. doi: 10.1172/JC199538 (2019).
Chen, J., Teo, B. H. D., Cai, Y., Wee, S. Y. K. & Lu, J. The linker histone H1.2 is a novel component of the nucleolar organizer regions. J Biol Chem 293, 2358-2369, doi:10.1074/jbc.M117.810184 (2018).
Coffman, R. L., Sher, A. & Seder, R. A. Vaccine adjuvants: putting innate immunity to work. Immunity 33, 492-503, doi:10.1016/j.immuni.2010.10.002 (2010).
Das, N. et al. HMGB1 Activates Proinflammatory Signaling via TLRS Leading to Allodynia. Cell Rep 17, 1128-1140, doi:10.1016/j.celrep.2016.09.076 (2016).
Degn, S. E. et al. Clonal Evolution of Autoreactive Germinal Centers. Cell 170, 913-926 e919, doi:10.1016/j.cell.2017.07.026 (2017).
de la Cruz, J., Karbstein, K. & Woolford, J. L., Jr. Functions of ribosomal proteins in assembly of eukaryotic ribosomes in vivo. Annu Rev Biochem 84, 93-129, doi:10.1146/annurev-biochem-060614-033917 (2015).
Derossi, D., Joliot, A. H., Chassaing, G. & Prochiantz, A. The third helix of the Antennapedia homeodomain translocates through biological membranes. J Biol Chem 269, 10444-10450 (1994).
Derossi, D. et al. Cell internalization of the third helix of the Antennapedia homeodomain is receptor-independent. J Biol Chem 271, 18188-18193, doi:10.1074/jbc.271.30.18188 (1996).
Duthie, M. S., Windish, H. P., Fox, C. B. & Reed, S. G. Use of defined TLR ligands as adjuvants within human vaccines. Immunol Rev 239, 178-196, doi:10.1111/j.1600-65X.2010.00978.x (2011).
Evans, B. C. et al. Ex vivo red blood cell hemolysis assay for the evaluation of pH-responsive endosomolytic agents for cytosolic delivery of biomacromolecular drugs. J Vis Exp, e50166, doi:10.3791/50166 (2013).
Grifoni, A. et al. A Sequence Homology and Bioinformatic Approach Can Predict Candidate Targets for Immune Responses to SARS-CoV-2. Cell Host Microbe 27, 671-680 e672, doi:10.1016/j.chom.2020.03.002 (2020).
Hirata, D. et al. Nucleolin as the earliest target molecule of autoantibodies produced in MRL/Ipr lupus-prone mice. Clin Immunol 97, 50-58, doi:10.1006/clim.2000.4916 (2000).
Hollingsworth, R E. and Jansen, K. Turning the Corner on Therapeutic Cancer Vaccines. NPJ Vaccines 4, 7, doi: 10.1038/s41541-019-0103-y (2019).
Kawai, T. & Akira, S. TLR signaling. Semin Immunol 19, 24-32, doi:10.1016/j.smim.2006.12.004 (2007).
Kawai, T. & Akira, S. The role of pattern-recognition receptors in innate immunity: update on Toll-like receptors. Nat Immunol 11, 373-384, doi:10.1038/ni.1863 (2010).
Lau, C. M. et al. RNA-associated autoantigens activate B cells by combined B cell antigen receptor/Toll-like receptor 7 engagement. J Exp Med 202, 1171-1177, doi:10.1084/jem.20050630 (2005).
Lee, S. & Nguyen, M. T. Recent advances of vaccine adjuvants for infectious diseases. Immune Netw 15, 51-57, doi:10.4110/in.2015.15.2.51 (2015).
Li, J. et al. Structural basis for the proinflammatory cytokine activity of high mobility group box 1. Mol Med 9, 37-45 (2003).
Lischwe, M. A., Smetana, K., Olson, M. O. & Busch, H. Proteins C23 and B23 are the major nucleolar silver staining proteins. Life Sci 25, 701-708, doi:10.1016/0024-3205(79)90512-5 (1979).
Maris, C., Dominguez, C. & Allain, F. H. The RNA recognition motif, a plastic RNA-binding platform to regulate post-transcriptional gene expression. FEBS J 272, 2118-2131, doi:10.1111/j.1742-4658.2005.04653.x (2005).
Mitchell, D. J., Kim, D. T., Steinman, L., Fathman, C. G. & Rothbard, J. B. Polyarginine enters cells more efficiently than other polycationic homopolymers. J Pept Res 56, 318-325, doi:10.1034/j.1399-3011.2000.00723.x (2000).
Mietzner, B. et al. Autoreactive IgG memory antibodies in patients with systemic lupus erythematosus arise from nonreactive and polyreactive precursors. Proc Natl Acad Sci USA 105, 9727-9732, doi:10.1073/pnas.0803644105 (2008).
Mevorach, D., Zhou, J. L., Song, X. & Elkon, K. B. Systemic exposure to irradiated apoptotic cells induces autoantibody production. J Exp Med 188, 387-392, doi:10.1084/jem.188.2.387 (1998).
Moore, M. W., Carbone, F. R., Bevan, M. J. Introduction of soluble protein into the class I pathway of antigen processing and presentation, Cell, 54(6): Pages 777-785 (1988).
Nakamura, R. M. & Tan, E. M. Recent progress in the study of autoantibodies to nuclear antigens. Hum Pathol 9, 85-91 (1978).
Nemazee, D. Mechanisms of central tolerance for B cells. Nat Rev Immunol 17, 281-294, doi:10.1038/nri.2017.19 (2017).
Poon, I. K., Lucas, C. D., Rossi, A. G. & Ravichandran, K. S. Apoptotic cell clearance: basic biology and therapeutic potential. Nat Rev Immunol 14, 166-180, doi:10.1038/nri3607 (2014).
Pulendran, B. & Ahmed, R. Translating innate immunity into immunological memory: implications for vaccine development. Cell 124, 849-863, doi:10.1016/j.cell.2006.02.019 (2006).
Rumore, P. M. & Steinman, C. R. Endogenous circulating DNA in systemic lupus erythematosus. Occurrence as multimeric complexes bound to histone. J Clin Invest 86, 69-74, doi:10.1172/JCI114716 (1990).
Savarese, E. et al. U1 small nuclear ribonucleoprotein immune complexes induce type 1 interferon in plasmacytoid dendritic cells through TLR7. Blood 107, 3229-3234, doi:10.1182/blood-2005-07-2650 (2006).
Sims, G. P., Rowe, D. C., Rietdijk, S. T., Herbst, R. & Coyle, A. J. HMGB1 and RAGE in inflammation and cancer. Annu Rev Immunol 28, 367-388, doi:10.1146/annurev.immunol.021908.132603 (2010).
Steinhagen, F., Kinjo, T., Bode, C. & Klinman, D. M. TLR-based immune adjuvants. Vaccine 29, 3341-3355, doi:10.1016/j.vaccine.2010.08.002 (2011).
Steinman, R. M. & Hemmi, H. Dendritic cells: translating innate to adaptive immunity. Curr Top Microbiol Immunol 311, 17-58, doi:10.1007/3-540-32636-7_2 (2006).
Suurmond, J. & Diamond, B. Autoantibodies in systemic autoimmune diseases: specificity and pathogenicity. J Clin Invest 125, 2194-2202, doi:10.1172/JCI78084 (2015).
Takeuchi, T. & Futaki, S. Current Understanding of Direct Translocation of Arginine-Rich Cell-Penetrating Peptides and Its Internalization Mechanisms. Chem Pharm Bull (Tokyo) 64, 1431-1437, doi:10.1248/cpb.c16-00505 (2016).
Tan, E. M., Schur, P. H., Carr, R. I. & Kunkel, H. G. Deoxybonucleic acid (DNA) and antibodies to DNA in the serum of patients with systemic lupus erythematosus. J Clin Invest 45, 1732-1740, doi:10.1172/JCI105479 (1966).
Thandapani, P., O'Connor, T. R., Bailey, T. L. & Richard, S. Defining the RGG/RG motif. Mol Cell 50, 613-623, doi:10.1016/j.molcel.2013.05.021 (2013).
Theofilopoulos, A. N., Kono, D. H. & Baccala, R. The multiple pathways to autoimmunity. Nat Immunol 18, 716-724, doi:10.1038/ni.3731 (2017).
Urbonaviciute, V. et al. Induction of inflammatory and immune responses by HMGB1-nucleosome complexes: implications for the pathogenesis of SLE. J Exp Med 205, 3007-3018, doi:10.1084/jem.20081165 (2008).
Vermeersch, P. & Bossuyt, X. Prevalence and clinical significance of rare antinuclear antibody patterns. Autoimmun Rev 12, 998-1003, doi:10.1016/j.autrev.2013.03.014 (2013).
Vives, E., Brodin, P. & Lebleu, B. A truncated HIV-1 Tat protein basic domain rapidly translocates through the plasma membrane and accumulates in the cell nucleus. J Biol Chem 272, 16010-16017, doi:10.1074/jbc.272.25.16010 (1997).
Wardemann, H. et al. Predominant autoantibody production by early human B cell precursors. Science 301, 1374-1377, doi:10.1126/science.1086907 (2003).
Wang, T. et al. High TLR7 Expression Drives the Expansion of CD19(+)CD24(hi)CD38(hi) Transitional B Cells and Autoantibody Production in SLE Patients. Front Immunol 10, 1243, doi:10.3389/fimmu.2019.01243 (2019).
Weindel, C. G. et al. B cell autophagy mediates TLR7-dependent autoimmunity and inflammation. Autophagy 11, 1010-1024, doi:10.1080/15548627.2015.1052206 (2015).
Welting, T. J., Raijmakers, R. & Pruijn, G. J. Autoantigenicity of nucleolar complexes. Autoimmunity reviews 2, 313-321 (2003).
Wu, S., Teo, B. H. D., Wee, S. Y. K., Chen, J. & Lu, J. The GAR/RGG motif defines a family of nuclear alarmins. Cell Death Dis 12, 477, doi:10.1038/s41419-021-03766-w (2021).
Zhang, H., Tay, P. N., Cao, W., Li, W. & Lu, J. Integrin-nucleated Toll-like receptor (TLR) dimerization reveals subcellular targeting of TLRs and distinct mechanisms of TLR4 activation and signaling. FEBS Lett 532, 171-176, doi:10.1016/s0014-5793(02)03669-4 (2002).
Zhang, J. et al. Polyreactive autoantibodies in systemic lupus erythematosus have pathogenic potential. J Autoimmun 33, 270-274, doi:10.1016/j.jaut.2009.03.011 (2009).

1. An isolated polypeptide comprising a glycine and arginine-rich (GAR/RGG) region with alarmin and/or cell penetrating activity.

2. The isolated peptide of claim 1, wherein the glycine and arginine-rich (GAR/RGG) region of the peptide comprises a plurality of amino acid trimers selected from the group consisting of RGG, GGR, FGG and GGF and/or tetramers selected from the group consisting of RGGG, GGGR, FGGG and GGGF.

3. The isolated polypeptide of claim 2, wherein the glycine and arginine-rich (GAR/RGG) region of the peptide further comprises tetramers selected from the group consisting of RGGG, GGGR, FGGG and GGGF and/or intervening amino acids selected from the group consisting of RG, GR, FR and GDR.

4. The isolated peptide of claim 1, wherein:
a) the peptide is selected from the group consisting of SEQ ID NO: 1, SEQ ID NO: 2, SEQ ID NO: 3, or an alarmin-active and/or cell-penetrating fragment or mutant thereof; and/or
b) the peptide comprises an amino acid sequence selected from the group consisting of SEQ ID NO: 4, SEQ ID NO: 5, SEQ ID NO: 6 and SEQ ID NO: 47, or an alarmin-active and/or cell-penetrating fragment or mutant thereof; and/or
c) the peptide mutant comprises an insertion of one or more ‘G’ residues within the GAR/RGG region to complete a triplet; and/or
d) the peptide or mutant thereof consists of an amino acid sequence selected from the group consisting of SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 20, SEQ ID NO: 21, SEQ ID NO: 22, SEQ ID NO: 23, SEQ ID NO: 24, SEQ ID NO: 53, SEQ ID NO: 54, SEQ ID NO: 55 and SEQ ID NO: 56.

5.-7. (canceled)

8. The isolated peptide of claim 1, wherein the peptide or mutant thereof has both alarmin activity and cell-penetrating activity.

9. The isolated peptide of claim 8, wherein the peptide consists of an amino acid sequence selected from the group consisting of SEQ ID NO: 7, SEQ ID NO: 8, SEQ ID NO: 12, SEQ ID NO: 13, SEQ ID NO: 14, SEQ ID NO: 53 and SEQ ID NO: 54.

10. The isolated peptide of claim 8, wherein the peptide has carrier function and consists of an amino acid sequence selected from the group consisting of SEQ ID NO: 21, SEQ ID NO: 23 and SEQ ID NO: 24.

11. An isolated fusion polypeptide comprising the isolated peptide of claim 1, fused to an antigen or cargo molecule.

12. The isolated fusion polypeptide of claim 11, wherein the peptide can penetrate cells and carry an antigen or cargo molecule into the cells; and/or
wherein the cells are dendritic cells or other antigen-presenting cells; and/or
wherein the at least one antigen is specific to a pathogen, such as a bacterium, fungus, parasite or virus, or to a cancer cell; and/or
wherein the cargo molecule is a drug or labelling molecule.

13.-15. (canceled)

16. A composition comprising:
a) the isolated peptide of claim 1 and at least one antigen; or
b) an isolated fusion polypeptide comprising the isolated peptide, fused to an antigen or cargo molecule, or
c) a cancer cell and at least one of the isolated peptide,
and one or more of a pharmaceutically acceptable excipient, diluent or carrier, or a mixture thereof.

17. A method of enhancing the immunogenicity of an antigen, wherein the antigen is specific to a pathogen, such as a bacterium, fungus, parasite or virus, or to a cancer cell, comprising
a) fusing an isolated alarmin-active and/or cell-penetrating peptide of claim 1 with the antigen; or
b) mixing the isolated alarmin-active and/or cell-penetrating peptide with the antigen.

18. (canceled)

19. A method of prophylaxis or treatment of a subject in need of such treatment, comprising administering to the body or cells of the subject:
a) the isolated alarmin-active and/or cell-penetrating peptide of claim 1, fused to or mixed with an antigen or cargo molecule; or
b) a composition comprising:
a) the isolated alarmin-active and/or cell-penetrating peptide and at least one antigen; or
b) an isolated fusion polypeptide comprising the isolated peptide, fused to an antigen or cargo molecule; or
c) a cancer cell and at least one of the isolated peptide;
and one or more of a pharmaceutically acceptable excipient, diluent or carrier, or a mixture thereof.

20. A method of activating at least one dendritic cell or other antigen presenting cell, or T cell, or cancer cell, comprising exposing the at least one dendritic cell, antigen presenting cell, or T cell, or cancer cell, to an isolated peptide of claim 1, or to the peptide fused to or mixed with an antigen or cargo molecule.

21. An isolated polynucleotide which encodes the peptide of claim 1 or encodes an isolated fusion polypeptide comprising the peptide.

22. A cloning or expression vector comprising one or more polynucleotides of claim 21.

23. A process for the production of the peptide of claim 1, or an isolated fusion polypeptide comprising the peptide, comprising:
culturing a host cell, or cell-free polypeptide manufacturing composition, comprising an expression vector comprising one or more polynucleotides that encodes the peptide or the isolated fusion polypeptide; and
isolating the peptide or fusion polypeptide.

24. A method of detecting GAR/RGG-containing peptides in a subject, comprising the steps;
i) providing a biological sample from the subject;
ii) determining a level of GAR/RGG-containing proteins present in the biological sample.

25. The method of claim 24, wherein the subject has an inflammatory disease, wherein a level of GAR/RGG-containing peptides above a control level indicates an inflammatory disease in the subject.

26. The method of claim 24, comprising contacting the biological sample from the subject with an antibody specific for a GAR/RGG region of the GAR/RGG-containing peptide, or bioactive GAR/RGG region mutants thereof.

27. The method of claim 24, wherein the biological sample is selected from the group consisting of blood, cerebrospinal fluid and urine.

28. A method of enhancing the intracellular delivery of an antigen or cargo molecule, for the purpose of research or disease treatment, comprising a combination of an alarmin-active and/or cell-penetrating peptide of claim 1 with the antigen or cargo molecule.
